(19) United States
(12) Patent Application Publication
(10) Pub. No.: US 2006/0270668 A1
(43) Pub. Date: Nov. 30, 2006

(54) Methods of synthesizing substituted 3-cyanoquinolines and intermediates thereof

(75) Inventors: Warren Chew, Outremont (CA); Gloria Karen Cheal, Beaconsfield (CA); Jacqueline Francesca Lunetta, Pierrefonds (CA)

Correspondence Address: FITZPATRICK CELLA (WYETH), 30 ROCKEFELLER PLAZA, NEW YORK, NY, 10112-3800, US

(73) Assignee: WYETH, Madison, NJ (US)

(21) Appl. No.: 11/439,984

(22) Filed: May 25, 2006

Related U.S. Application Data
(60) Provisional application No. 60/684,391, filed on May 25, 2005.

(51) Int. Cl. A61K 31/5377; A61K 31/506; A61K 31/496; A61K 31/4709; C07D 413/02; C07D 403/02

(52) U.S. Cl. 514/232.5; 514/256; 514/252.06; 514/312; 514/313; 544/126; 544/333; 544/363; 546/153; 546/159

(57) ABSTRACT

The invention is directed to methods of making substituted 3-cyanoquinolines, including compounds according to the following formula: 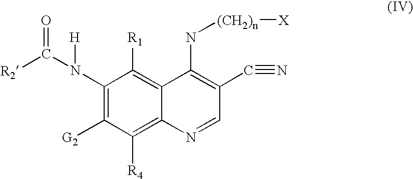 The methods are amenable to large scale manufacture, avoid the use of chromatographic separations, and provide stable, high purity product more efficiently than in the prior art.

This application claims the benefit of priority of U.S. Provisional Patent Application No. 60/684,391, filed May 25, 2005, incorporated herein by reference.

### BACKGROUND OF THE INVENTION

1. Field of the Invention

This invention relates to methods of making certain substituted 3-cyanoquinoline compounds as well as the pharmaceutically acceptable salts thereof. The compounds made by the methods of the present invention may inhibit the action of certain growth factor receptor protein tyrosine kinases (PTK) and other protein kinases thereby inhibiting the abnormal growth of certain cell types. The compounds made by the methods may therefore be useful for the treatment of certain diseases that are the result of deregulation of these PTKs and find utility, for example, in treatment of cancer in mammals. The methods herein have been adapted for large-scale synthesis.

2. Related Background Art

Protein kinases are a class of enzymes that catalyze the transfer of a phosphate group from ATP to a tyrosine, serine, threonine, or histidine residue located on a protein substrate, many of which play a role in normal cell growth. Correspondingly, several growth factor receptor proteins function as protein tyrosine kinases (PTKs) to effect signaling and are known as receptor tyrosine kinases (RTKs).

The RTKs comprise one of the larger families of PTKs and have diverse biological activity.

At present, at least nineteen (19) distinct subfamilies of RTKs have been identified. One such subfamily is the “HER” family of RTKs, which includes EGFR (epithelial growth factor receptor), HER2, HER3 and HER4. It has been shown that under certain conditions, as a result of either mutation or over expression, these RTKs can become deregulated; the result of which is uncontrolled cell proliferation which can lead to tumor growth and cancer. Wilks, A. F., Adv. Cancer Res., 60, 43 (1993) and Parsons, J. T.; Parsons, S. J., Important Advances in Oncology, DeVita, V. T. Ed., J. B. Lippincott Co., Phila., 3 (1993). For example, over expression of the receptor kinase product of the erbB-2 oncogene has been associated with human breast and ovarian cancers. Slamon, D. J. et al., Science, 244, 707 (1989) and Science, 235, 177 (1987). In addition, deregulation of EGFR kinase has been associated with epidermoid tumors. Reiss, M., et al., Cancer Res., 51, 6254 (1991), breast tumors (Macias, A. et al., Anticancer Res., 7, 459 (1987)), and tumors involving other major organs (Gullick, W. J., Brit. Med. Bull., 47, 87 (1991)). RTK inhibitors, therefore have potential therapeutic value for the treatment of cancer and other diseases characterized by uncontrolled or abnormal cell growth. Accordingly, many recent studies have dealt with the development of specific RTK inhibitors as potential anti-cancer therapeutic agents. Some recent reviews include: Traxler, P., Exp. Opin. Ther. Patents, 8, 1599 (1998) and Bridges, A. J., Emerging Drugs, 3, 279 (1998).

U.S. Pat. Nos. 6,002,008, 6,288,082, and 6,297,258, to Wissner et al., and U.S. Pat. No. 6,384,051 to Frost et al., describe certain substituted 3-cyanoquinolines, methods of making them and their biological activity. The disclosures of these patents are incorporated by reference herein in their entirety. More efficient methods of synthesis, particularly for large scale synthesis, would be highly desirable.

### SUMMARY OF THE INVENTION

The invention is directed to methods of making compounds according to the schemes, formulas and definitions below. The methods are amenable to large scale manufacture, in some cases avoid the use of chromatographic separations, and provide high purity product more efficiently than in the prior art.

In one aspect, the invention is a method for preparing substituted 3-cyanoquinolines comprising the step of reacting

(i) a compound of formula H-Z-(CH₂)ₙ—X, and

(ii) a 3-cyanoquinoline intermediate having formula (Ia) 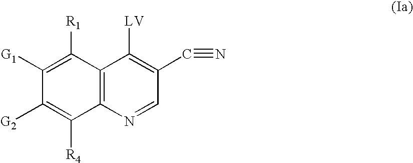
in the presence of a catalytic effective amount of an acid catalyst to produce a compound of formula (IIa)

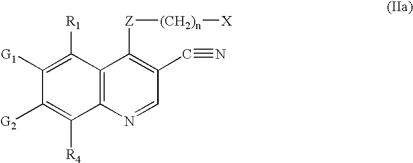

wherein X is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S-S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkyl amino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or

X is cycloalkyl of 3 to 7 carbon atoms, which may be optionally substituted with one or more alkyl of 1 to 6 carbon atom groups; or

X is a pyridinyl, pyrimidinyl, or phenyl ring, wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino; or

X is a radical having the formula:

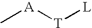

wherein A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;

T is bonded to a carbon of A and is:
—NH(CH₂)ₘ—, —O(CH₂)ₘ—, —S(CH₂)ₘ—, —NR(CH₂)ₘ—, —(CH₂)ₘ—, —(CH₂)ₘNH—, —(CH₂)ₘO—, —(CH₂)ₘS—, or —(CH₂)ₘNR—;

L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of3-10 carbon atoms, N-alkylaminoalkoxy of 2-9 carbon atoms, N,N-dialkylaminoalkoxy of 3-10 carbon atoms, mercapto, and benzoylamino; or

L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;

LV is a leaving group,

Z is —NH—, —O—, —S—, or —NR—,

R is alkyl of 1-6 carbon atoms,

G₁, G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 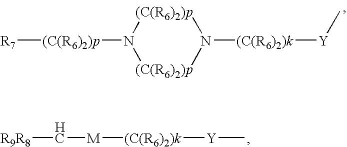
R₇—(C(R₆)₂)_{g}—Y—, R₇—(C(R₆)₂)ₚ-M-(C(R₆)₂)ₖ—Y—, or Het-(C(R₆)₂)_{q}—W—(C(R₆)₂—Y—; or optionally

G₁ and/or G₂ are independently selected from protected amino group and R₂—NH—;

Y is a divalent radical selected from the group consisting of

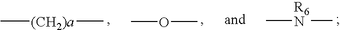

R₇ is —NR₆R₆, —OR₆, -J, —N(R₆)₃⁺, or —NR₆(OR₆);

M is >NR₆, —O—, >N—(C(R₆)₂)ₚNR₆R₆, or >N—(C(R₆)₂)ₚ—R₆;

W is >NR₆, or is a bond;

Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and

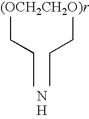

wherein Het is optionally mono- or di-substituted on carbon or nitrogen with R₆, optionally mono- or di-substituted on carbon with hydroxy, —N(R₆)₂, or

—OR₆, optionally mono or di-substituted on carbon with the mono-valent radicals —(C(R₆)₂)ₛOR₆ or —(C(R₆)₂)ₛN(R₆)₂, and optionally mono or di-substituted on a saturated carbon with divalent radicals —O— or —O(C(R₆)₂)ₛO—;

R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl of 2-7 carbon atoms, phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;

R₂ is selected from the group consisting of acetyl, t-BOC, CBZ,

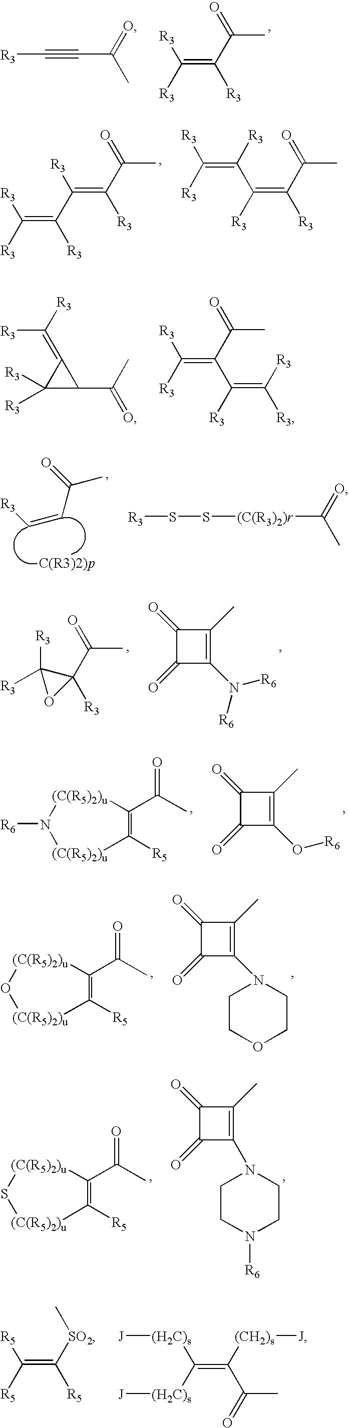

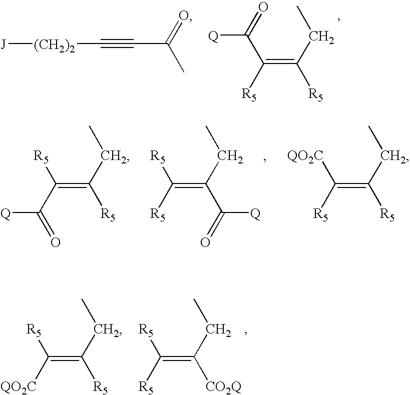

R₃ is independently hydrogen, alkyl of 1-6 carbon atoms, aminoalkyl of 1-6 carbon atoms, cycloaminoalkyl of 4-12 carbon atoms, carboxy, carboalkoxy of 1-6 carbon atoms, phenyl, carboalkyl of 2-7 carbon atoms, 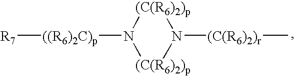
R₇—(C(R₆)₂)ₛ—, R₇—(C(R₆)₂)ₚ-M-(C(R₇)₂)ᵣ—, R₈R₉—CH-M-(C(R₆)₂)ᵣ—, or Het-(C(R₆)₂)_{q}—W—(C(R₆)₂)ᵣ—;

R₅ is independently hydrogen, alkyl of 1-6 carbon atoms, carboxy, carboalkoxy of 1-6 carbon atoms, phenyl, carboalkyl of 2-7 carbon atoms, 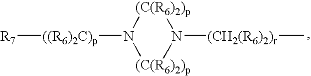
R₇—(C(R₆)₂)ₛ—, R₇—(C(R₆)₂)ₚ-M-(C(R₆)₂)ᵣ—, R₈R₉—CH-M-(C(R₆)₂)ᵣ—, or Het-(C(R₆)₂)_{q}—W—(C(R₆)₂)ᵣ—;

R₈, and R₉ are each, independently, —(C(R₆)₂)ᵣNR₆R₆, or —(C(R₆)₂)ᵣOR₆;

J is independently hydrogen, chlorine, fluorine, or bromine;

Q is alkyl of 1-6 carbon atoms or hydrogen;

a=0 or 1;

g=1-6;

k=0-4;

m is 0-3;

n is 0-1,

p=2-4;

q=0-4;

r=1-4;

s=1-6;

u=0-4 and v=0-4, wherein the sum of u+v is 2-4.

In another embodiment, G₁ is a protected amine selected from the group consisting of acetamides (including without limitation trifluoroacetamide), benzamide, cyclic imides (including, without limitation, phthalimide, maleimide, and 2,5-dimethylpyrrole), tert-butoxycarbonyl (t-BOC) protected amine and benzyloxycarbonyl (CBZ) protected amine.

In yet another aspect of the invention, the methods for preparing 4-amino-3-cyanoquinolines according to the invention comprise the step of reacting

(i) a compound of formula H₂N—(CH₂)ₙ—X, and

(ii) a 3-cyanoquinoline starting material having formula (I)

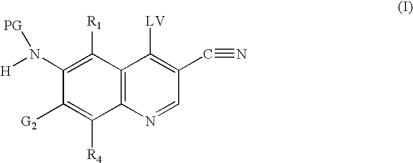

in the presence of a catalytic effective amount of an acid catalyst to produce a 4-amino-3-cyanoquinoline having formula (II) 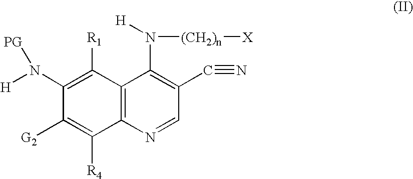
wherein n, X, R₁, R₄, and G₂ are defined as above, LV is chloro, iodo bromo, alkylsulfonate, or the like, and wherein PG is a protecting group, such as t-BOC, CBZ, or acyl.

In still another aspect, compounds produced according to the methods of the invention are recrystallized to form a salt, such as a maleate salt.

In this aspect, a method of synthesizing substituted 3-cyanoquinolines according to the invention may comprise the steps of:

reacting an activated carboxylate of formula (VI) 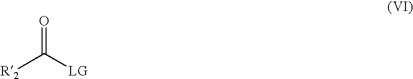
with an intermediate of formula (III′) 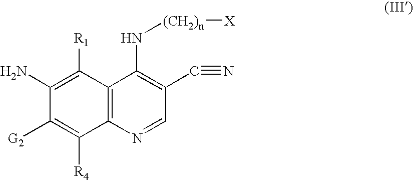
to form a compound of formula (VII) 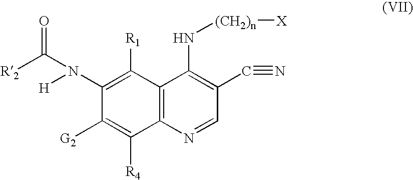
and

recrystallizing said compound (VII) from a mixture of said compound (VII) in a solvent to form a salt of said compound, wherein

LG is a leaving group selected such that the activated carboxylate of Formula (IV) is a halide, anhydride (eg. isobutylchloroformate), acyl azide, 1,3,5-triazine, aromatic boronic acid, Lawesson's reagent, or peptide-type coupling reagent including, without limitation, DCC, TiCl₄, activated phosphates, Sn[N(TMS)₂]₂, N-halosuccinimide/ph₃P, Cl₃CCN/Ph₃P, (R₂N)₂Mg, SO₂ClF, chlorosulfonyl isocyanide, TsCl/base, metal alkoxides, PyBOP, BOP, and EDCI/HOBt.

R′₂ is alkyl of 1-6 carbon atoms, optionally mono or di-substituted with amino groups or cycloamino groups, or R′₂ is alkenyl of 2-6 carbon atoms optionally mono or di-substituted with amino groups or cycloamino groups, and wherein

X is a pyridinyl, pyrimidinyl, or phenyl ring, wherein the pyridinyl, pyrimidinyl, or phenyl ring optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino; or
X is a radical having the formula: 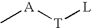
wherein A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
T is bonded to a carbon of A and is:
—NH(CH₂)ₘ—, —(CH₂)ₘ—, —S(CH₂)ₘ—, —NR(CH₂)ₘ—, —(CH₂)ₘ—, —(CH₂)ₘNH—, —(CH₂)ₘO—, —(CH₂)ₘS—, or —(CH₂)ₘNR—;
L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
and wherein G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 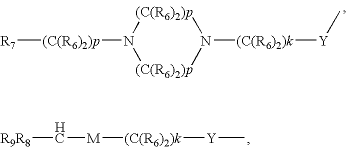
   R₇—(C(R₆)₂)_{g}—Y—, R₇—(C(R₆)₂)ₚ-M-(C(R₆)₂)ₖ—Y—, or Het-(C(R₆)₂)_{q}W—(C(R₆)₂—Y—; or
R₁ and R₄ are as defined above and G₂ is R₂—NH—;
Y is a divalent radical selected from the group consisting of 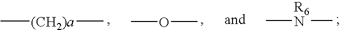
R₇ is —NR₆R₆, —OR₆, -J, —N(R₆)₃⁺, or —NR₆(OR₆);
M is >NR₆, —O—, >N—(C(R₆)₂)ₚNR₆R₆, or >N—(C(R₆)₂)ₚ—OR₆;
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 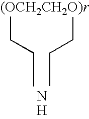
wherein Het is optionally mono- or di-substituted on carbon or nitrogen with R₆, optionally mono- or di-substituted on carbon with hydroxy, —N(R₆)₂, or —OR₆, optionally mono or di-substituted on carbon with the mono-valent radicals —(C(R₆)₂)ₛOR₆ or —(C(R₆)₂)ₛN(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂ is selected from the group consisting of 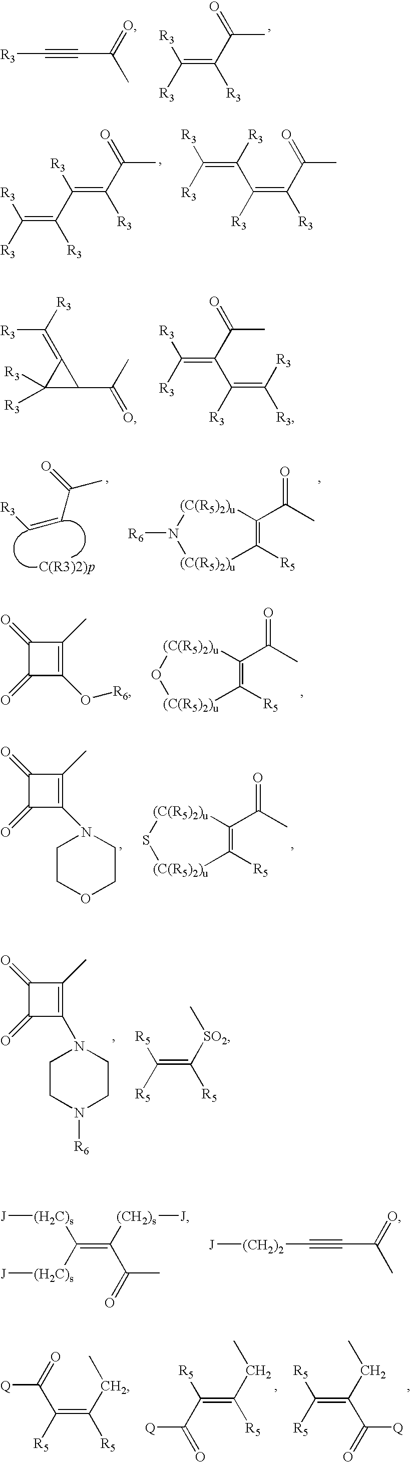 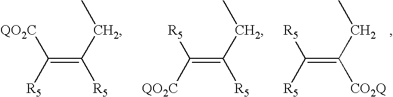
R₃ is independently hydrogen, alkyl of 1-6 carbon atoms, aminoalkyl of 1-6 carbon atoms, cycloaminoalkyl of 4-12 carbon atoms, carboxy, carboalkoxy of 1-6 carbon atoms, phenyl, carboalkyl of 2-7 carbon atoms, 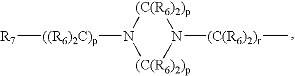
   R₇—(C(R₆)₂)ₛ—, R₇—(C(R₆)₂)ₚ-M-(C(R₇)₂)ᵣ—, R₈R₉—CH-M-(C(R₆)₂)ᵣ—, or Het-(C(R₆)₂)_{q}—W—(C(R₆)₂)ᵣ—;
R₅ is independently hydrogen, alkyl of 1-6 carbon atoms, carboxy, carboalkoxy of 1-6 carbon atoms, phenyl, carboalkyl of 2-7 carbon atoms, 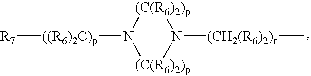
   R₇—(C(R₆)₂)ₛ—, R₇—(C(R₆)₂)ₚ-M-(C(R₆)₂)ᵣ—, R₈R₉—CH-M-(C(R₆)₂)ᵣ—, or Het-(C(R₆)₂)_{q}—W—(C(R₆)₂)ᵣ—;
R₈, and R₉ are each, independently, —(C(R₆)₂)ᵣNR₆R₆, or —(C(R₆)₂)ᵣOR₆;
J is independently hydrogen, chlorine, fluorine, or bromine;
Q is alkyl of 1-6 carbon atoms or hydrogen;
a=0 or 1;
g=1-6;
k=0-4;
m is 0-3;
n is 0-1,
p=2-4;
q=0-4;
r=1-4;
s=1-6;
u=0-4 and v=0-4, wherein the sum of u+v is 2-4.

In embodiments, R′₂ in Formula (VII) above is a 4-(dimethylamino)-2-butenyl radical, a 4-(piperidino)-2-buteneyl radical, a 4-(pyrrolidino)-2-butenyl radical, or a 3,4-(dipyrrolidino)-2-butenyl radical.

In another aspect, the invention includes a telescoped reaction sequence for preparing compounds according to the above schemes and definitions, in which the reaction intermediates are not isolated before performing the next reaction step.

### BRIEF DESCRIPTION OF THE FIGURE

FIG. 1 shows a DSC thermogram of (E)-N-{4-[3-chloro-4-(2-pyridinylmethoxy)anilino]-3-cyano-7-ethoxy-6-quinolinyl}-4-(dimethylamino)-2-buteneamide maleate.

### DETAILED DESCRIPTION OF THE INVENTION

### DEFINITIONS

For purposes of this invention the term “alkyl,” unless stated otherwise, includes both straight and branched alkyl moieties which can contain as many as 12 carbon atoms. Preferably, the alkyl moiety contains between 1 to 6 carbon atoms, though 1 to 4 carbon atoms is more preferable. The term “alkenyl” refers to a radical aliphatic hydrocarbon containing at least one double bond and includes both straight and branched alkenyl moieties of 2 to 6 carbon atoms. Such alkenyl moieties may exist in the E or Z configurations; the compounds of this invention include both configurations. The term “alkynyl” includes both straight chain and branched moieties containing 2 to 6 carbon atoms having at least one triple bond. The term “cycloalkyl” refers to alicyclic hydrocarbon groups having 3 to 12 carbon atoms and includes but is not limited to: cyclopropyl, cyclobutyl, cyclopentyl, cyclohexyl, cycloheptyl, norbornyl, or adamantyl.

For purposes of this invention the term “aryl” is defined as an aromatic hydrocarbon moiety and may be substituted or unsubstituted. An aryl group preferably contains 6 to 12 carbon atoms and may be selected from, but not limited to, the group: phenyl, α-naphthyl, β-naphthyl, biphenyl, anthryl, tetrahydronaphthyl, phenanthryl, fluorenyl, indanyl, biphenylenyl, acenaphthenyl, acenaphthylenyl, or phenanthrenyl groups. An aryl group may be optionally mono-, di-, tri- or tetra-substituted with substituents selected from, but not limited to, the group consisting of alkyl, acyl, alkoxycarbonyl, alkoxy, alkoxyalkyl, alkoxyalkoxy, cyano, halogen, hydroxy, nitro, trifluoromethyl, trifluoromethoxy, trifluoropropyl, amino, alkylamino, dialkylamino, dialkylaminoalkyl, hydroxyalkyl, alkoxyalkyl, alkylthio, —SO₃H, —SO₂NH₂, —SO₂NH(alkyl), —SO₂N(alkyl)₂ , —CO₂H, CO₂NH₂, CO₂NH(alkyl), and —CO₂N(alkyl)₂. Preferred substituents for aryl and heteroaryl include: alkyl, halogen, amino, alkylamino, dialkylamino, trifluoromethyl, trifluoromethoxy, arylalkyl, and alkylaryl.

For purposes of this invention the term “heteroaryl” is defined as an aromatic heterocyclic ring system (monocyclic or bicyclic) where the heteroaryl moieties are five or six membered rings containing 1 to 4 heteroatoms selected from the group consisting of S, N, and O, and include but is not limited to: (1) furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, pyrrolidinyl; (2) a bicyclic aromatic heterocycle where a phenyl, pyridine, pyrimidine or pyridizine ring is: (i) fused to a 6-membered aromatic (unsaturated) heterocyclic ring having at least one heteroatom; (ii) fused to a 5-membered aromatic or nonaromatic (unsaturated) heterocyclic ring having at least one heteroatom selected from O, N or S. Preferably a bicyclic heteroaryl group contains 8 to 12 carbon atoms. Preferred substituents for heteroaryl include: alkyl, halogen, amino, alkylamino, dialkylamino, trifluoromethyl, trifluoromethoxy, arylalkyl, and alkylaryl.

For the purposes of this invention the term “alkoxy” is defined as C₁-C₆-alkyl-O—; the term “aryloxy” is defined as aryl-O—; the term “heteroaryloxy” is defined as heteroaryl-O—; wherein alkyl, aryl, and heteroaryl are as defined above.

For purposes of this invention the term “arylalkyl” is defined as aryl-C₁-C₆-alkyl-; arylalkyl moieties include benzyl, 1-phenylethyl, 2-phenylethyl, 3-phenylpropyl, 2-phenylpropyl and the like.

For purposes of this invention the term “alkanoyloxymethyl” is defined as —CH₂OC(O)R, wherein R is alkyl of 1 to 6 carbon atoms.

For purposes of this invention the term “alkylthio” is defined as C₁-C₆-alkyl-S.

For purposes of this invention “alkylthioalkyl,” and “aryloxyalkyl,” denote an alkyl group as defined above that is further substituted with an alkoxy or alkylthio as defined above.

The terms “alkylamino” and “dialkylamino” refer to moieties with one or two alkyl groups wherein the alkyl chain is 1 to 6 carbons and the groups may be the same or different. The terms “monoalkylaminoalkyl” and “dialkylaminoalkyl” refer to monoalkylamino and dialkylamino moieties with one or two alkyl groups (the same or different) bonded to the nitrogen atom which is attached to an alkyl group of 1 to 6 carbon atoms. Preferably a dialkylaminoalkyl moiety consist of 3 to 10 carbon atoms and a alkylaminoalkyl moiety consist of from 2 to 9 carbon atoms.

The terms “alkylaminoalkoxy” and “dialkylaminoalkoxy” refer to alkylamino and dialkylamino moieties with one or two alkyl groups (the same or different) bonded to the nitrogen atom which is attached to an alkoxy group of 1 to 6 carbon atoms. Preferably a dialkylaminoalkoxy moiety consist of 3 to 10 carbon atoms and a alkylaminoalkoxy moiety consist of from 2 to 9 carbon atoms.

For purposes of this invention the term “benzoylamino” is defined as a Ph-OC(O)NH— moiety.

For purposes of this invention the term “carboxy” is defined as a —COOH moiety.

For purposes of this invention the term “alkanoylamino” is defined as a —NH—COOR moiety, wherein R is alkyl of 1 to 6 carbon atoms.

For purposes of this invention the term “alkenoylamino” and “alkynoylamino” are defined as a —NH—COOR moiety, wherein R is alkenyl or alkynyl of 3 to 8 carbon atoms.

For purposes of this invention the term “carboalkoxy” is defined as —CO₂R, wherein R is alkyl of 1 to 6 carbon atoms.

For purposes of this invention the term “carboalkyl” is defined as —COR, wherein R is alkyl of 1 to 6 carbon atoms.

For purposes of this invention the term “carboxyalkyl” is defined as a HOOCR— moiety, wherein R is alkyl of 1 to 6 carbon atoms.

For purposes of this invention the term “carboalkoxyalkyl” is defined as a —R—CO₂—R′ moiety, wherein R and R′ are alkyl and together consist of from 2 to 7 carbon atoms.

For purposes of this invention the term “aminoalkyl” is defined as H₂N-alkyl, wherein the alkyl group consist of 1 to 5 carbon atoms.

“Azido” is a radical of the formula —N₃.

“Acyl” is an organic radical derived from a carboxylic acid. Preferred examples include but are not limited to, acetyl, propionyl, trifluoroacetyl and benzoyl.

For purposes of this invention the term “alkylsulfinyl” is defined as a R′SO— radical, where R′ is an alkyl radical of 1 to 6 carbon atoms. Alkylsulfonyl is a R′SO₂— radical, where R′ is an alkyl radical of 1 to 6 carbon atoms. Alkylsulfonamido, alkenylsulfonamido, alkynylsulfonamido are R′SO₂NH— radicals, where R′ is an alkyl radical of 1 to 6 carbon atoms, an alkenyl radical of 2 to 6 carbon atoms, or an alkynyl radical of 2 to 6 carbon atoms, respectively.

Saturated or partially saturated heteroaryl groups are defined in this invention as heterocyclic rings selected from but not limited to the moieties: azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl.

The term “substituent” is used herein to refer to an atom radical, a functional group radical or a moiety radical that replaces a hydrogen radical on a molecule. Unless expressly stated otherwise, it should be assumed that any of the substituents may be optionally substituted with one or more groups selected from: alkyl, halogen, haloalkyl, hydroxyalkyl, nitro, amino, hydroxy, cyano, alkylamino, dialkylamino, alkoxy, haloalkoxy, alkoxyalkyl, alkoxyalkoxy, oxo, alkylthio, mercapto, haloalkylthio, aryl, aryloxy, arylthio, heteroaryl, heteroaryloxy, heteroarylthio, acyl, —CO₂-alkyl, —SO₃H, —SO₂NH₂, —SO₂NH-alkyl, —SO₂NH—(alkyl)₂, —CO₂H, —CO₂NH₂, —CO₂NH-alkyl and —CO₂N-(alkyl)₂.

For the purposes of this invention the term “substituted” refers to where a hydrogen radical on a molecule has been replaced by another atom radical, a functional group radical or a moiety radical; these radicals being generally referred to as “substituents.”

The term “protecting group” (PG) refers to a group introduced into a molecule to protect a sensitive functional group or specific position on the molecule from reacting when the molecule is exposed to reagents or conditions to transform or react another part of the molecule. Thereafter the protecting group can be removed. A “protected group” is the sensitive functional group together with the protecting moiety. Suitable protecting groups are well known in the art and include acid-labile, base-labile, photoremovable, or removable under neutral conditions. See, e.g., Green, Protecting Groups in Organic Synthesis, Wiley 1991, 2^{nd} ed., pp. 309-405, which is incorporated herein by reference. Such protecting groups include, without limitation, acetyl, tert-butoxycarboxyl and benzyloxycarbonyl. In some instances, an amino group is protected. Exemplary protected amino groups include acetamides, benzamides, cyclic imides (e.g., phthalimide, maleimide, 2,3-dichloromaleimide, succinemide, dihydrophthalimide), pyrroles (e.g. 2,5-dimethylpyrrole), tert-butoxycarbonyl protected amine and benzyloxycarbonyl protected amide. However, as used herein, a “protected amino group” does not include a urea group or a protected urea group. Protecting group (PG) does not include ureas or protected ureas and does not, together with the group that is being protected, form a urea group or a protected urea group.

Except where specifically defined, the term “leaving group” (LV or LG) means any group that is the conjugate base of an acid which can be displaced by a desired group in the course of a reaction. Good leaving groups include, without limitation, chloro, iodo and bromo, alkylsulfonates such as methanesulfonates, and aryl sulfonates, such as methyl benzenesulfonate, ethyl p-toluenesulfonate, and the like.

The compounds of this invention may contain an asymmetric carbon atom and may thus give rise to stereoisomers, such as enantiomers and diastereomers. The stereioisomers of the instant invention are named according to the Cahn-Ingold-Prelog System. While shown without respect to stereochemistry in formulas (I) and (II), the present invention includes all-the individual possible stereoisomers; as well as the racemic mixtures and other mixtures of R and S stereoisomers (scalemic mixtures which are mixtures of unequal amounts of enantiomers) and prodrugs and pharmaceutically acceptable salts thereof. It should be noted that stereoisomers of the invention having the same relative configuration at a chiral center may nevertheless have different R and S designations depending on the substitution at the indicated chiral center.

Ac, if not otherwise defined, means acyl.

ACN means acetonitrile.

Ar, if not otherwise defined, means aryl.

BOP means benzotriazol-1-yloxy-tris(dimethylamino)phosphonium hexafluorophosphate.

DMF means dimethylformamide.

DSC means differential scanning calorimetry.

EtOH means ethanol.

EtOAc means ethyl acetate.

IPA means isopropanol.

HPLC means high performance liquid chromatography.

MEK means methyl ethyl ketone.

MIBK means methyl iso-butylketone.

MeOH means methanol.

MeSO₃H means methanesulfonic acid.

MTBK means methyl t-butylketone.

NMP means N-methylpyrrolidone.

n-PrOH means n-propanol.

n-BuOH means n-butanol.

PyBOB means benzotriazole-1-yl-oxy-tris-pyrrolidino-phosphonium hexafluorophosphate, (benzotriazol-1-yloxy)tripyrrolidinophosphonium PF₆.

THF means tetrahydrofuran.

Synthesis

In one aspect, the invention is a method for preparing a substituted 3-cyanoquinoline having the following formula:

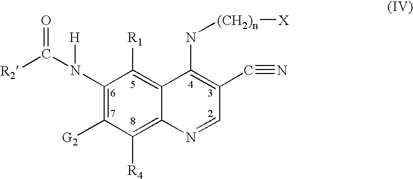

In an intermediate step, an intermediate having the formula H₂N—(CH₂)ₙ—X is reacted with a 3-cyanoquinoline provided with a leaving group at the 4 position and a protecting group at the 6 position. The reaction is preferably performed by heating the reactants together in alcohol over a period of time (e.g. 4-6 hours) to form the 4-substituted compound. It has been found that to initiate the reaction at a large scale, it is necessary to add a catalytic amount of acid catalyst, defined as an amount sufficient to render the reaction mixture acidic. The effective amount therefore depends on factors including the particular acid catalyst used and on the pH of the reaction mixture. Pyridine hydrochloride has been used effectively in amounts of about 1.16 eq. Methanesulfonic acid has been used effectively in amounts of about 0.025 eq. Suitable acid catalysts include pyridine hydrochloride, hydrochloric acid, sulfuric acid, acetic acid, trifluoroacetic acid, phosphoric acid, p-toluenesulfonic acid and methanesulfonic acid. Methanesulfonic acid is the most preferred. An effective amount of acid catalyst usually ranges between about 0.025 eq. and 1.2 eq.

Preferably, amine intermediate H₂N—(CH₂)ₙ—X is an aniline, wherein n is 0, and X is an optionally substituted phenyl ring as defined above. This aniline intermediate may be produced by reducing a compound of formula X—NO₂, for example by hydrogenation.

In embodiments, particular aniline intermediates are formed by reacting a nitroaryl or nitroheteroaryl of formula AR—NO₂ with a compound of formula AR′—CH₂—OH, in the presence of base and a suitable solvent, such as DMF, ACN or THF, followed by a catalytic hydrogenation of the resulting nitro compound using platinum on carbon. In this instance, AR and AR′ both independently denote aryl, heteroaryl, or substituted aryl or heteroaryl. Thus, particular aniline intermediates include aryloxyanilines, which may be formed, for example, by reacting pyridyl carbinol with a chloro substituted nitrobenzene to form 3-chloro-4-(pyridylmethoxy)aniline. Other suitable aryloxyaniline intermediates include 3-chloro-4-(benzyloxy)aniline, 3-chloro-4-(fluorobenzyloxy)aniline, and 3-chloro-4-(thiophenyl)aniline, which can be synthesized in an analogous manner.

Alternatively, the aniline intermediates can be formed by reacting a hydroxynitroaryl or hydroxynitroheteroaryl of formula HO—AR—NO₂ with compound of formula AR′—CH₂-LV′ in the presence of base and suitable solvent, such as DMF, ACN or THF, followed by catalytic hydrogenation of the resulting nitro compound using platinum on carbon, wherein AR and AR′ both independently denote aryl, heteroaryl or substituted aryl or heteroaryl. LV′ denotes a leaving group that can be displaced by the hydroxynitroaryl or hydroxynitroheteroaryl. The leaving groups are typically the conjugate base anion of a strong acid, such as chloro, bromo, iodo, mesylate, tosylate or triflate. The preferred leaving groups are chloro and bromo.

Preferably, the aniline intermediate is formed by catalytic hydrogenation, and the reaction product of this step is not completely isolated before performing the above-described acid catalyzed coupling reaction. This is referred to herein as a “telescoped” reaction sequence.

In embodiments, the starting 3-cyanoquinoline for the above coupling reaction has the following formula I:

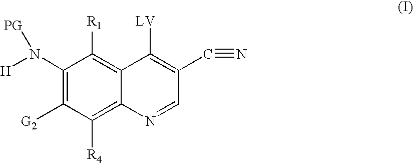

LV is any leaving group that can be displaced by the aniline intermediate at the 4 position. Leaving groups are typically the conjugate base anion of a strong acid, such as chloro, bromo, iodo, mesylate, tosylate or triflate groups. In embodiments LV is selected from the group consisting of chloro iodo and bromo. The preferred leaving group is chloro. PG is a protecting group for the amino nitrogen at the 6 position of the quinoline moiety, preferably acetyl, tert-butoxycarbonyl (t-BOC) or benyloxycarbonyl (CBZ); or PG together with the amine that PG is attached to forms a trifluoroacetamide group, a benzamide group, or a cyclic imide group, such as phthalimide, maleimide, 2,5-dimethylpyrrole, or the like. The above-described coupling may be followed by hydrolyzing the amide at the 6 position to form a second intermediate aniline compound. In a preferred embodiment, the hydrolysis is advantageously conducted in the presence of HCl and water. R₁, G₂ and R₄ are defined as above. In preferred embodiments, R₁ and R₄ are hydrogen and G₂ is alkoxy.

Preferably, the coupling and the hydrolysis are “telescoped,” that is, conducted in sequence without completely isolating the intermediate reaction product from an earlier step. Hydrolysis produces an acid salt which may be converted to free base, as described in detail in the Examples.

In another embodiment, a side chain is attached at the 6 position of the quinoline core by reacting the quinoline core with an activated carboxylate of formula R′₂—(C═O)-LG wherein LG is chloro or —O(C═O)-alkyl. Thus, the activated carboxylate derived from the corresponding carboxylic acid is, without limitation, an acid chloride, mixed anhydride, an activated ester or an activated group facilitated by peptide-type coupling reagents or other amidation catalysts, wherein R′₂ is any organic moiety such that after the coupling of the side chain is completed, the 6 position of the resulting compound is defined according to G₁ above. In preferred embodiments R₂′ may be, for example, alkyl of 1-6 carbon atoms, optionally mono or di-substituted with amino groups or cycloamino groups, or R₂′ may be alkenyl of 2-6 carbon atoms, optionally mono or di-substituted with amino groups or cycloamino groups. In another preferred embodiment, the activated carboxylate is an acid chloride or mixed anhydride.

Preferably, for large-scale production, the steps of (a) hydrogenating the nitroaryl compound to prepare a first aniline intermediate, (b) coupling the first aniline intermediate with a 3-cyanoquinoline core, (c) deprotecting the quinoline to form a second aniline intermediate, and (d) preparing the free base of a second aniline intermediate, can be telescoped so that intermediate reaction products from steps (a) through (c) are not completely isolated, but reacted substantially “as is” in the next reaction sequence.

A general scheme showing the sequence of these steps is shown in Scheme 1.

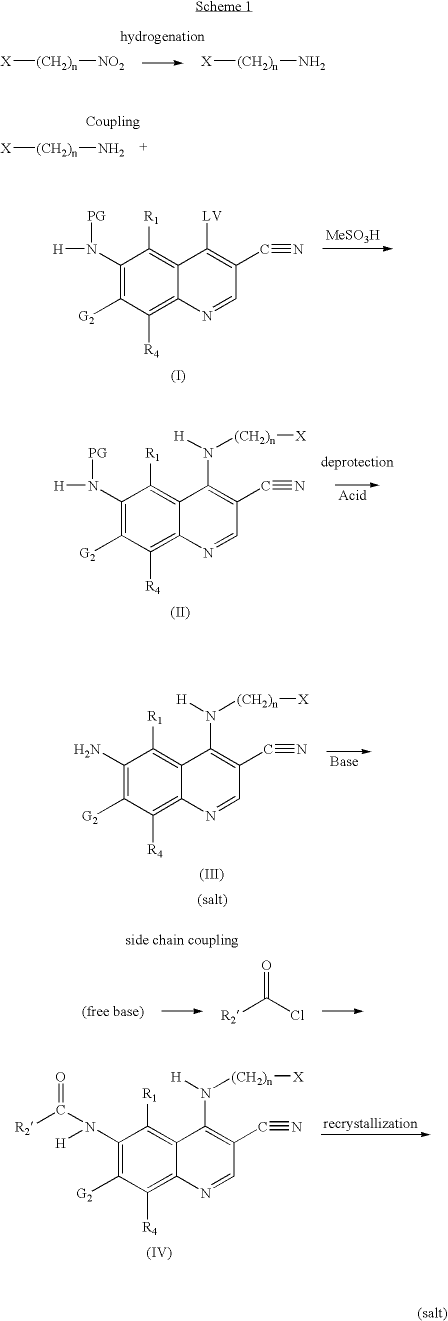

Instead of an acid chloride, shown in Scheme 1, a mixed anhydride or an activated carboxylate derived from the corresponding carboxylic acid may be used. The preferred mode of activation is via the acid chloride or mixed anhydride.

Specific examples with preferred starting materials according to the invention are shown in Scheme 2 and Scheme 3.

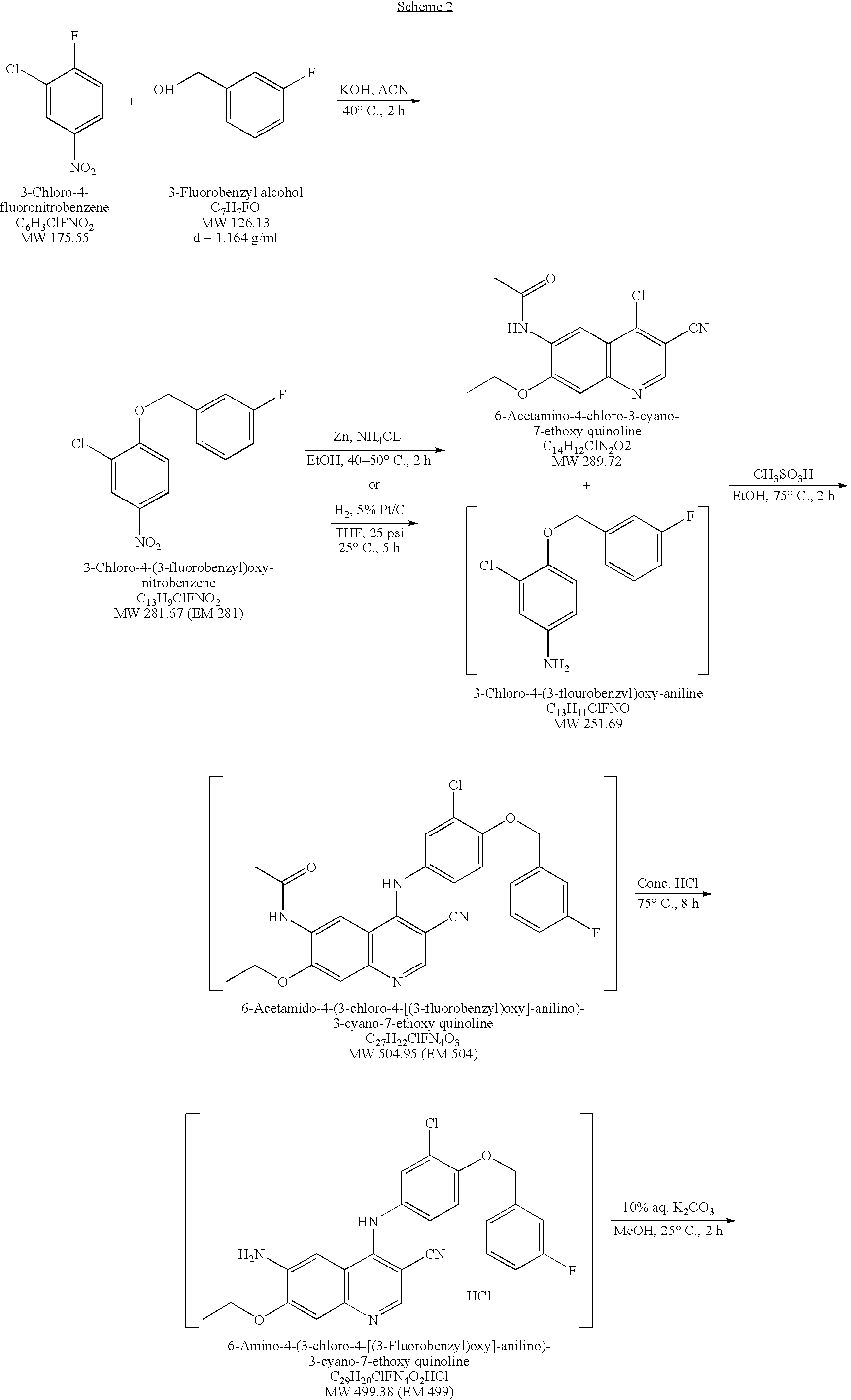

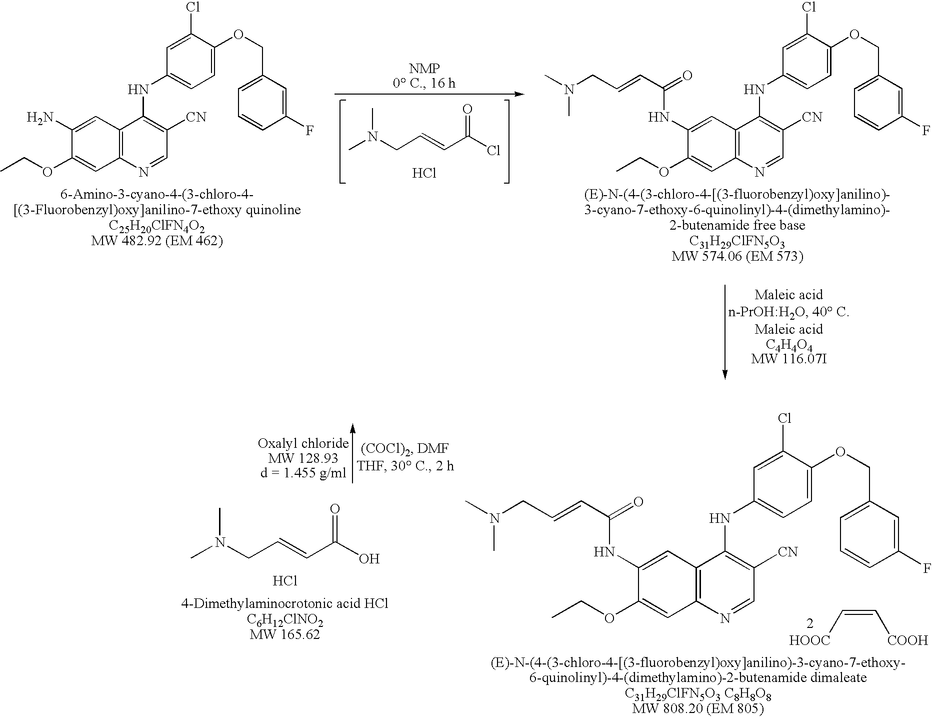

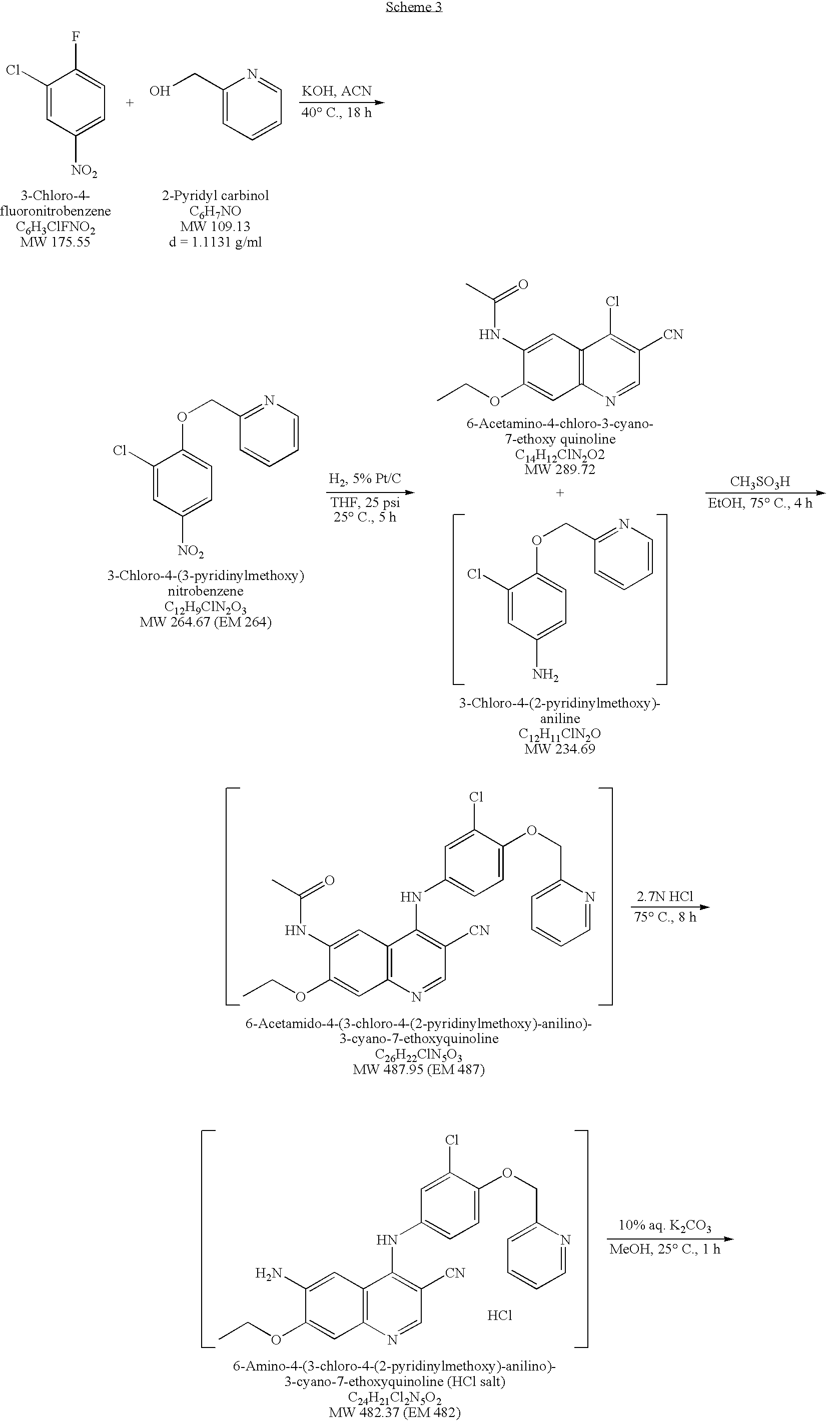

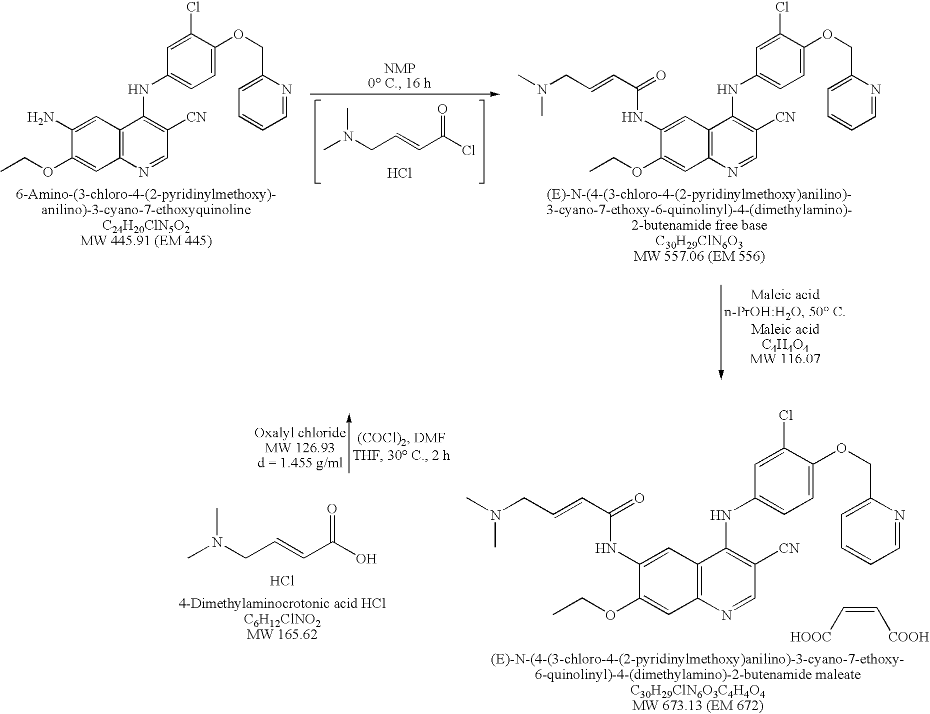

Reaction of AR—NO₂ with AR′—CH₂—OH followed by hydrogenation to form a first aniline intermediate is described in Examples 1 and 2 below in connection with the formation of a specific aniline intermediate, 3-chloro-4-(2-pyridylmethoxy)aniline (Scheme 3). Analogous syntheses of 3-chloro-[4-(3-fluorobenzyloxy)aniline (Scheme 2), and 3-chloro-4-(2-pyridylmethoxy)aniline, are described in Examples 1a, 2a and 1b, 2b respectively.

### EXAMPLE 1

### Synthesis of 3-chloro-4-(2-pyridylmethoxy)nitrobenzene

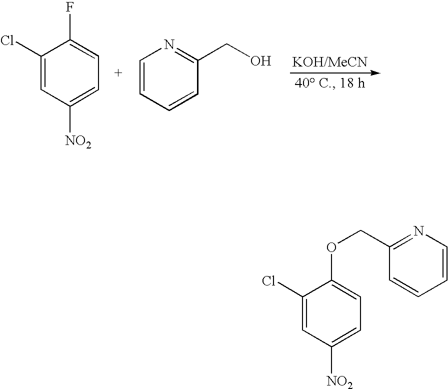

2-pyridinyl carbinol (31.08 g, 1.05 eq) was dissolved in ACN (750 mL) and KOH flakes (85%) were added (20.6 g, 1.25 eq.). The resulting suspension was warmed to 35° C. A solution of the 3-chloro-4-fluoronitrobenzene (50.0 g, 0.285 mol) in ACN (250 mL) was added at 35-40° C. The mixture was held for 14 hours. The mixture was then cooled back to 20-25° C., quenched with H₂O (1 L) and the resulting slurry filtered and washed with H₂O (3×100 mL). The resulting product was isolated as a tan solid in 93% yield with a greater than 99.5% purity as determined by HPLC area.

### EXAMPLE 1a

To accomplish the analogous synthesis of 3-chloro-4-(3-fluorobenzyloxy)nitrobenzene, 3-fluorobenzyl alcohol (0.30 kg, 2.39 mole, 1.05 eq) was dissolved in ACN (6.0 L) and to it was added potassium hydroxide flakes (85%) (0.16 kg, 1.25 eq). The resulting suspension was warmed to 35° C. A solution of the 3-chloro-4-fluoronitrobenzene (0.40 kg, 2.28 mol) in ACN (2.0 L) was added at 35-40° C. The mixture was held for 18 hours. The mixture was then cooled back to 20-25° C., quenched with water (8 L) and the resulting slurry filtered and washed with water (2×0.40 L). The resulting product was dried at 45° C., under 10 mm Hg pressure, for 25 hours to give 0.59 kg (92% yield).

### EXAMPLE 1b

To prepare 4-(benzyloxy)3-chloronitrobenzene, benzyl alcohol (0.34 kg, 3.14 mole, 1.10 eq) was dissolved in acetonitrile (1.70 L) and to it was added potassium hydroxide flakes (85%) (0.24 kg, 1.50 eq). The resulting suspension was warmed to 25° C. A solution of the 3-chloro-4-fluoronitrobenzene (0.50 kg, 2.85 mol, 1.0 eq) in acetonitrile (0.75 L) was added keeping the pot temperature<45° C. The mixture was held for 14 h. The mixture was then cooled back to 0-15° C., quenched with water (2.5 L) and the resulting slurry was filtered and washed with water (2×0.50 L). The resulting product was dried at 50° C., under 10 mm Hg pressure, for 24 hours to give 0.73 kg (97% yield).

Experimental results for the reaction of Example 1 with different bases and solvents are shown in Table 1. The last three entries on Table 1 are large scale runs in which a 5% excess of pyridyl carbinol was used.

TABLE 1
Preparation of Nitroaryl Intermediate

| Scale | | Vol- | | Base | Time | Temp | Yield | Purity |
|---|---|---|---|---|---|---|---|---|
| (g) | Solvent | umes | Base | Eq. | (h) | (° C.) | (%) | (%) |
| 2.0 | DMF | 20 | KOH | 1.1 | 20 | RT | 90.5 | 94.7 |
| 2.0 | NMP | 10 | NaH | 1.2 | 20 | RT | 48.7 | 78.4 |
| 2.0 | ACN | 20 | KOH | 1.1 | 4 | RT | 93.2 | 98.4 |
| 2.0 | EtOAc | 10 | KOH | 1.1 | 72 | RT | NA | NA |
| 10.0 | DMF | 15 | KOH | 1.1 | 23 | RT | 76.5 | 96.7 |
| | | | | | 4 | 35 | | |
| 10.0 | ACN | 15 | KOH | 1.1 | 23 | RT | 91.8 | 99.4 |
| 2.0 | THF | 20 | KOH | 1.1 | 20 | RT | 87.5 | 99.2 |
| 2.0 | DMF | 20 | K₂CO₃ | 1.0 | 26 | RT | 81.9 | 98.5 |
| | | | extra | | 3 | 40 | | |
| | | | 2.0eqK₂CO₃ | | 3 | 40 | | |
| 2.0 | ACN | 20 | K₂CO₃ | 1.0 | 18 | RT | NA | NA |
| | | | | | 3 | 40 | | |
| 2.0 | THF | 20 | K₂CO₃ | 1.0 | 18 | RT | NA | NA |
| 50.0 | ACN | 20 | KOH | 1.1 | 20 | 40 | 93.5 | 99.8 |
| 200 | ACN | 20 | KOH | 1.1 | 16 | 40 | 86.0 | 97.6 |
| 200 | ACN | 20 | KOH | 1.25 | 16 | 40 | 93.5 | 96.9 |
| 400 | ACN | 20 | KOH | 1.25 | 16 | 40 | 91.5 | 98.4 |
| 400 | ACN | 20 | KOH | 1.25 | 16 | 40 | 93.8 | 98.1 |

NA = not applicable
RT = room temperature (20-25° C.)

### EXAMPLE 2

Preparation of 3-chloro-4-(2-pyridylmethoxy)aniline from the nitrobenzene product of Example 1 was accomplished with catalytic hydrogenation using platinum on carbon.

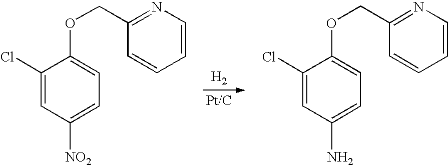

A typical hydrogenation was done using 6 volumes of THF, 2% by weight of 5% Pt/C (50% water wet), at 25 psi and at 25-30° C. for approximately 4-6 hours. The reaction is slightly exothermic and the temperature will rise to about 30-35° C. Cooling is necessary to maintain the temperature below 30° C.

As a specific example, a mixture of 3-chloro-4-(2-pyridylmethoxy)nitrobenzene (0.15 kg, 0.57 mole) and 2% (w/w) of 5% Pt/C (6.0 g) in tetrahydrofuran (0.90 L) was hydrogenated at 25 psi for at least 5 hours. The mixture was filtered through a celite pad and washed with tetrahydrofuran (0.60 L). The filtrate was distilled to a volume of about 0.75 L and ethanol (1.12 L) was added. Distillation was continued to a volume of about 0.75 L and ethanol (2.85 L) was added. The mixture may be used “as is” in the step of Example 3 below.

### EXAMPLE 2a

To accomplish an analogous synthesis of 3-chloro-4-(3-fluorobenzyloxy)aniline, zinc (0.464 kg) was added to a mixture of 3-chloro-4-(3-fluorobenzyloxy)nitrobenzene (0.40 kg, 1.42 mole) and ethanol (4.0 L). The mixture was heated to 40-50° C. A solution of ammonium chloride (0.152 kg) in water (0.80 L) was added over 0.5 hour keeping the pot temperature at 40-50° C. The mixture was stirred for 2 hours, filtered and washed with hot (40-50° C.) ethanol (2×0.40 L). The filtrate was distilled to a volume of about 0.80 L and 2-methyltetrahydrofuran (2.0 L) was added to dissolve the product. Water (0.80 L) and saturated brine (0.40 L) were added and the layers separated. The organic layer was washed with water (0.60 L), and distilled to a volume of about 0.40 L. Ethanol (2.0 L) was added and distillation continued to a volume of 1.2 L.

### EXAMPLE 2b

To prepare 4-(benzyloxy)-3-chloroaniline, a mixture of 4-(benzyloxy)-3-chloronitrobenzene (0.325 kg, 1.23 mole, 1.0 eq) and 1% (w/w) of 5% Pt/C (3.25 g) in isopropanol (3.25 L) was hydrogenated at 25 psi for a minimum of 4.5 h. The mixture was filtered through a celite pad and washed with isopropanol (2.0 L). The filtrates were used as is in the next step.

Performing the hydrogenation in isopropyl alcohol (IPA), methanol (MeOH), or ethanol (EtOH) may result in the product being contaminated with late eluting impurity that partially precipitates out on standing in solution. It was found that performing the hydrogenation in a solvent where both the product and starting material are soluble, such as tetrahydrofuran (THF), resulted in greater product purity and required much less solvent. Thus, THF is a preferred solvent for this step. Experimental results showing the effect of different reaction conditions are shown in Table 2. For the larger scale runs, the first aniline intermediate was not isolated (“NI”) before proceeding with the next step.

TABLE 2
Hydrogenation to Form First Aniline Intermediate

| | 5% | | | | |
|---|---|---|---|---|---|
| Scale (g) | Pt/C** | Solvent | Vol | Time (h) | Yield (%) |
| 2.0 | 1 | IPA | 50 | 3 | 79.6 |
| | | | | 18 | |
| 2.0 | 5 | EtOH | 60 | 3 | 100* |
| 10 | 1 | THF | 10 | 4 | 94.5 |
| | | | | 7 | |
| 10 | 1 | EtOH | 10 | 3 | 95.6 |
| 30 | 1.05 | THF | 6.5 | 12 | 96.3 |
| | | | | 14 | |
| 100 | 2 | THF | 6 | 4.5 | 97.1 |
| 400 | 2 | THF | 6 | 4 | NI |
| 500 | 2 | THF | 6 | 4 | NI |
| 100 | 2 | THF | 6 | 5 | NI |
| 150 | 2 | THF | 6 | 5 | NI |
| | | | | 7 | |

*Solid impurities noted after reaction completion.
**percent by weight of starting material.

### EXAMPLE 3

Following hydrogenation to form the first aniline intermediate, acid catalyzed coupling was performed to prepare 4-[3-chloro-4-(2-pyridylmethoxy)anilino]-3-cyano-7-ethoxy-6-N-acetylaminoquinoline, as shown below:

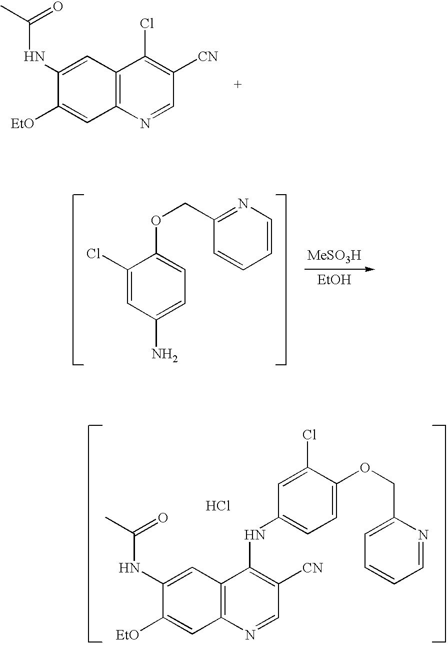

To perform the coupling reaction, the two reactants were heated together in alcohol at 65-78° C. over 4-6 hours, yielding the product. The reaction begins as an amber slurry and thickens to a lighter beige slurry as it approaches completion. Upon scaling up from 75 g to 350 g, it proved necessary to add a catalytic amount (0.025 eq.) of methanesulfonic acid to initiate the reaction. As a specific example, 4-chloro-3-cyano-7-ethoxy-6-N-acetylaminoquinoline (0.141 kg, 0.49 mole) was added to the mixture of Example 2, followed by ethanol (0.037 L) to give a suspension. A catalytic amount of methanesulfonic acid (1.17 g) was added at 20-25° C. The resulting slurry was heated to 70-75° C. and held for a minimum of 4 hours. Thickening of the slurry was evident after 1.5 hours. Following reaction completion, the mixture was cooled to room temperature and may be used “as is” in the telescoped reaction of Example 4 below.

### EXAMPLE 3a

To prepare 6-acetamido-4-[3-chloro-4-(3-fluorobenzyloxy)anilino]-3-cyano-7-ethoxyquinoline, ethanol (4.80 L) was added to the aniline solution followed by 4-chloro-3-cyano-7-ethoxy-6-N-acetylaminoquinoline (0.350 kg, 1.11 mole). A catalytic amount of methanesulfonic acid (2.0 ml) was added at 20-25° C. The resulting suspension was heated to 70-75° C. and held for a minimum of 2 h. Thickening of the slurry was evident during this holding period. Following reaction completion, the mixture was used as is in the following telescoped reaction.

### EXAMPLE 3b

To prepare 6-acetamido-4-[4-(benzyloxy)-3-chloroanilino]-3-cyano-7-ethoxy-quinoline, isopropanol (6.75 L) was added to the aniline solution followed by 4-chloro-3-cyano-7-ethoxy-6-N-acetylaminoquinoline (0.277 kg, 0.96 mole, 0.78 eq). A catalytic amount of methane sulfonic acid (3.50 ml) was added at 20-25° C. The resulting suspension was heated to 80-85° C. and held for a minimum of 10 hr. Thickening of the slurry was evident during this holding period. Following reaction completion, the mixture was cooled to 25-35° C., filtered and the cake washed with isopropanol (3×0.25 L). The cake was used as is in the following telescoped reaction.

As solvents EtOH, DMF or other suitable solvent may be used. Experimental results obtained using different solvents and reaction conditions are shown in Table 3. Difficulty filtering the product of this step (noted in several entries on Table 3) was circumvented by not isolating the solid at this point, but telescoping the reaction with the next step. It has been found that on the order of 20 volumes of EtOH were necessary to achieve reasonable stirring, but that the reaction can proceed in only 10 volumes of DMF, without significant loss in purity.

In Table 3, where the entry is labelled NI, the intermediate product was not isolated, but carried into the next reaction step.

TABLE 3
Coupling Reaction

| | Coupling | Temp | Time | Yield | |
|---|---|---|---|---|---|
| Solvent | Solvent | (° C.) | (h) | (%) | Comments |
| IPA | EtOH | 78 | 4 | 85.4 | contains impurity |
| THF | EtOH | 78 | 4 | 90.5 | v. slow filtration |
| THF | THF | 68 | 4 | NA | Only 16% product formed |
| THF | EtOH | 78 | 4 | 94.2 | v. slow filtration |
| EtOH | IPA | 82 | 5 | NA | No reaction |
| EtOH | MeOH | 65 | 5 | 60.0 | v. slow filtration |
| THF | EtOH | 78 | 1.5 | 80.3 | v. slow filtration |
| | (MeSO₃H) | | | | |
| THF | EtOH | 78 | 4 | 86.0 | v. slow filtration |
| THF | EtOH | 78 | 3 | 85.7 | 4 h filtration - hard, green |
| | (MeSO₃H) | | | | coated solid on drying |
| THF | Dimethoxy | 85 | 2 | 74.2 | Faster filtration (<1 hr) |
| | ethane | | | | Nice yellow solid |
| THF | Diethoxy | 85 | 5 | — | — |
| | Methane | | | | |
| THF | Dimethoxy | 70 | 6 | — | — |
| | Ethane | | | | |
| THF | EtOH | 78 | 6 | 96.6 | Slow filtration |
| THF | DMF | 78 | 0.5 | 65.6 | Some product lost in filtrate |
| | (MeSO₃H) | | | | |
| THF | DMF | 70 | 8 | NI | See Note 1 |
| | (MeSO₃H) | | | | |
| THF | EtOH | 78 | 6 | ND | See Note 2 |
| | (MeSO₃H) | | | | |
| THF | EtOH | 78 | 4 | NI | Yield to the free base is |
| | (MeSO₃H) | | | | 80.4%^{3/} |
| THF | EtOH | 75 | 4 | NI | Yield to the free base is |
| | (MeSO₃H) | | | | 83%^{3/} |
| THF | EtOH | 75 | 4 | NI | Yield to the free base is |
| | (MeSO₃H) | | | | 86%^{3/} |

NR = no reaction,
NI = not isolated;
ND = not determined;
NA = not available
1. Carried through to the deprotection and generation of free base to give 69.5% overall yield.
2. The overall yield after the deprotection and generation of the free base is 76.1%
³This reaction was not filtered at all but taken as slurry to the next step.

### EXAMPLE 4

### Deprotection

The deprotection of the quinoline intermediate formed by the coupling reaction using 2N HCl in water is preferred as noted in Table 4 below. As in the previous Examples, the intermediate product of this step is advantageously not isolated, but carried over as a wet cake into the next step.

Preparation of 4-[3-chloro-4-(2-pyridylmethoxy)anilino]-3-cyano-7-ethoxy-6-aminoquinoline hydrochloride.

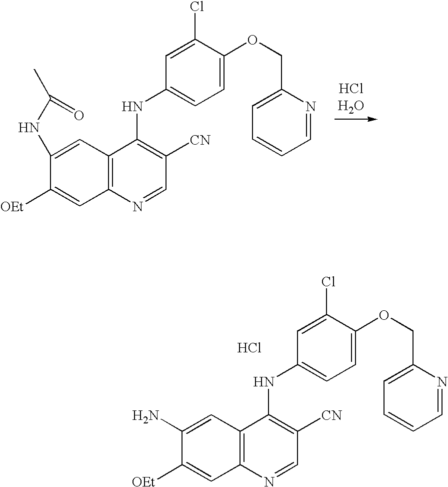

The reaction mixture from the previous step (Example 3) was taken as is and to it was added 2.7N HCl (3.3 L) in H₂O (16.0 L). The slurry was heated to 70° C. and held for 19 hours. The resulting slurry was then filtered and rinsed with 1:1 EtOH/H₂O (4×1.0 L). The product was isolated as a wet cake and carried through to the next step. A small sample was dried at this stage and analyzed. The HCl salt had a strength of 98.9%.

### EXAMPLE 4a

To prepare 6-amino-4-[3-chloro-4-(3-fluorobenzyloxy)anilino]-3-cyano-7-ethoxyquinoline hydrochloride, the reaction mixture from the previous step was taken as is and to it was added ethanol (1.6 L) and concentrated hydrochloric acid (1.38 L) to bring the pH to 1-3. The suspension was held at 70-75° C. for a minimum of 2 h. After 1 h, the mixture thickens and ethanol (0.80 L) was added. After 2 h, water (6.80 L) was added, the mixture stirred for 1 h and then cooled to 35-45° C. and stirred overnight (12 h). The mixture was filtered and rinsed with 1:1 ethanol/water (2×0.84 L) at 35-45° C. The product was isolated as a wet cake and carried through to the next step.

### EXAMPLE 4b

To prepare 6-amino-4-[4-(benzyloxy)-3-chloroanilino]-3-cyano-7-ethoxyquinoline hydrochloride, the wet cake from the previous step was taken as is and to it was added a 2 N solution of concentrated hydrochloric acid (1.16 L) in methanol (5.84 L). The suspension was heated to 63-68° C. and held for a minimum of 30 h. The mixture was cooled to 20-30° C., filtered and rinsed with methanol (2×0.30 L). The product was isolated as a wet cake and carried through to the next step.

TABLE 4
Deprotection

| | Reagent/ | | | | |
|---|---|---|---|---|---|
| | Solvent | | Time | | |
| Scale (g) | (vols) | Temp (° C.) | (h) | Yield (%) | Purity |
| 1.0 | 2N HCl | 50 | 1 | No reaction | NA |
| | EtOH (5) | | | after 1 hour | |
| 1.0 | 2N HCl | 60 | 1 | 87.5 | 97.6% str |
| | H₂O (50) | | | | 1.3% SM |
| 3.0 | 2N HCl | 85 | 1 | 97.0 | 97.8% str |
| | H₂O (30) | | | | 1.7% SM |
| 10.0 | 2N HCl | 85 | 3 | 95 | 99.0% |
| | H₂O (25) | | | | 0.3% SM |
| 10.0 | 2N HCl | 85 | 4 | 90.3 | 98.7% |
| | H₂O (25) | | | | 0.4% SM |
| 634 | 2N HCl | 85 | 4 | ND | 98.7% str |
| | H₂O (25) | | | | 0.3% SM |
| 795 | 2N HCl | 70 | 19 | ND | 98.8% str |
| | H₂O (25) | | | | 0.09% SM |
| 184 | 8.4N HCl | 70 | 20 | ND | NA |
| | H₂O (2) | | 44 | | |

ND = not determined (the product was used in the next step as a wet cake)
NA = not available
SM = starting material

### EXAMPLE 5

### Preparation of Free Base

The 4-[3-chloro-4-(2-pyridylmethoxy)anilino]-3-cyano-7-ethoxy-6-aminoquinoline HCl salt was converted to the corresponding free base by treatment with 10% potassium carbonate (1.8 L) in MeOH (2.82 L). The mixture was stirred for a minimum of 2.5 hours and the pH was 9-10. The product was filtered, washed with 1:1 methanol/water (3×0.19 L) and dried (at 45-50° C. at a pressure of 10 mm Hg, for 24 hours) to give 0.186 kg of product with an overall yield of 86% over 4 steps.

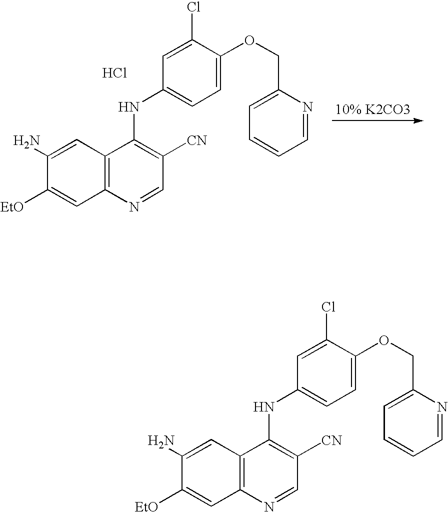

### EXAMPLE 5a

To prepare 6-amino-4-[3-chloro-4-(3-fluorobenzyloxy)anilino]-3-cyano-7-ethoxyquinoline free base, the 6-amino-4-[3-chloro-4-(3-fluorobenzyloxy)anilino]-3-cyano-7-ethoxyquinoline hydrochloride salt was converted to its corresponding free base by treatment with 10% potassium carbonate (0.22 kg in 2.27 L water) in methanol (7.21 L) until pH was 10. The mixture was stirred for a minimum of 2 h. The beige suspension was filtered, washed with 1:1 methanol/water (2×0.84 L) and dried (45-50° C., 10 mm Hg, 24 h) to give 0.51 kg of product with an overall yield of 99% over 4 steps.

### EXAMPLE 5b

To prepare 6-amino-4-[4-(benzyloxy)-3-chloroanilino]-3-cyano-7-ethoxyquinoline free base, the 6-amino-4-[4-(benzyloxy)-3-chloroanilino]-3-cyano-7-ethoxyquinoline hydrochloride salt was converted to its corresponding free base by treatment with 10% aqueous potassium carbonate (0.213 kg in 2.13 L) in methanol (6.40 L). The mixture was stirred for a minimum of 1.5 h keeping the pH at 9-10. The product was filtered, washed with water (2×0.50 L) and dried (50-60° C., 10 mm Hg, 20 h) to give 0.347 kg of product with an overall yield of 82% over 4 steps.

### EXAMPLE 6

### Side Chain Coupling

An acid chloride of formula R′₂—(C═O)—Cl, a mixed anhydride or an activated carboxylate R′2-(C═O)-LG derived from the corresponding carboxylic acid, may be used to couple a side chain at the 6 position to form a 6-amido-4-amino-3 cyanoquinoline. R′₂ may be alkyl of 1-6 carbon atoms, which may be mono- or di-substituted with amino groups or cycloamino groups, or R′₂ may be alkenyl of 2-6 carbon atoms which may be mono- or di-substituted with amino groups or cycloamino groups.

Using the 2-step sequence shown below, an activated carboxylate is prepared in situ and coupled with the aniline. Although the acid chloride can be prepared in acetonitile, a better yield was obtained when the acid chloride was prepared in THF. In both cases, the aniline should be dissolved in NMP before amidation. It is believed that formation of product is better due to better solubility of the aniline in a THF/NMP mixture rather than in an ACN/NMP combination.

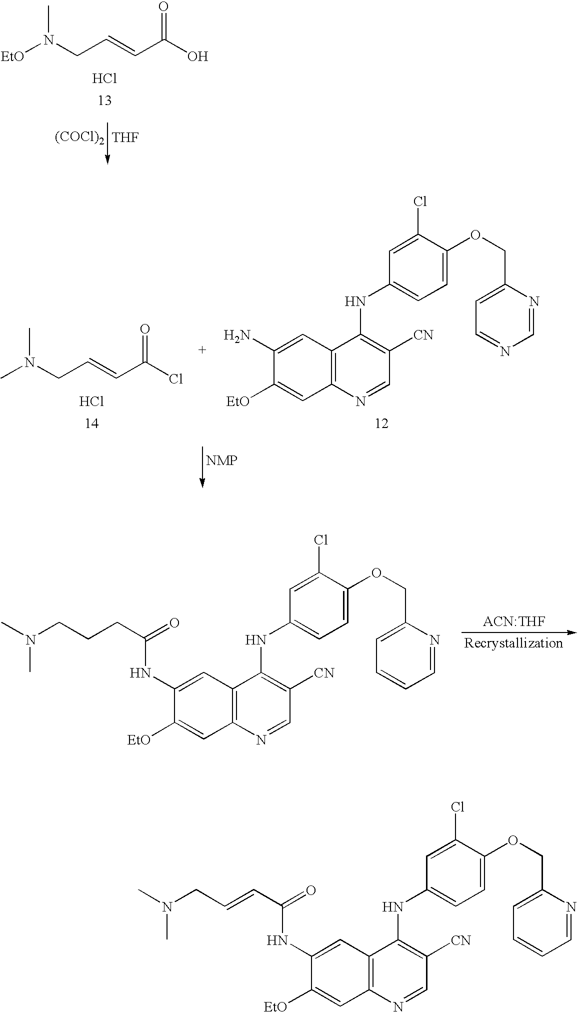

The amount of 4-N,N-dimethylaminocrotonic acid needed was 2 equivalents with respect to aniline. A slight undercharge of 1.95 eq of oxalyl chloride was added along with a catalytic amount (3 mol %) of DMF. The acid chloride was formed via the Vilsmeier intermediate. The completion test for the acid chloride reaction consists of quenching an aliquot of the reaction into ethanol and detecting by HPLC the crotonic acid ethyl ester. This method serves as a check to ensure complete consumption of oxalyl chloride. Excess oxalyl chloride will form diethyl oxalate when quenched in ethanol.

The acid chloride is stable after holding for up to 5 hours at 0-10° C., when decomposition begins. After 20 hours, complete decomposition takes place. If the acid chloride is allowed to warm, decomposition occurs and its effectiveness is diminished.

The quality of the starting crotonic acid also plays a role in this coupling reaction, as commercially available crotonic acid may contain acetic acid. Acetic acid is detrimental to this reaction. 6-N-acetyl quinoline can be formed which is difficult to remove from the final product. The acetic acid can be removed by re-slurrying the crotonic acid in 4 volumes of isopropanol at room tempature, filtering and drying preferably to a level of less than 0.01%.

It was found that the addition of the aniline solution in NMP to the acid chloride gave a better yield as compared to adding the acid chloride to the aniline. The addition is done keeping the temperature at 0-5° C. The coupling reaction is slow and requires holding overnight at this temperature. It is not desirable to raise the reaction temperature as the stability of the acid chloride diminishes.

The reaction is quenched using aqueous sodium hydroxide at 40° C. and then filtered at that temperature. Quenching the reaction at 40° C. gives bigger crystals that are easily filterable. It was observed that filtration at 40° C. was faster than at room temperature. The product is recrystallized from a 1.5:1 mixture of acetonitrile:THF (15 volumes) at 70-75° C. This in-process purification beneficially removes unreacted aniline. The recovery yields are typically greater than 85%.

To demonstrate a specific synthesis of (E)-N-{4-[3-chloro-4-(2-pyridinylmethoxy)anilino]-3-cyano-7-ethoxy-6-quinolinyl}-4-(dimethylamino)-2-butenamide, a solution of 4-N,N-dimethylaminocrotonic acid hydrochloride (186 g, 1.12 mol) in THF (1.88 L) and a catalytic amount of DMF (2 mL) was cooled to 0-5° C. Oxalyl chloride (97 mL, 1.09 mol, 0.95 eq) was added dropwise over 45 minutes. The mixture was then warmed to 25-30° C. and stirred for 2 hours. The yellow solution was checked for complete consumption of oxalyl chloride by HPLC, then cooled to 0-5° C.

When the reaction is deemed complete, a solution of 4-[4-(2-pyridylmethoxy)-3-chloro]amino-6-amino-3-cyano-7-ethoxyquinoline (250 g, 0.56 mol) in N-methyl-2-pyrolidinone (1.88 L) was added dropwise over 2 hours keeping the temperature at 0-5° C. The mixture was stirred for at least 3 hours until less than about 2% of the starting aniline remains by HPLC, which takes about 3 hours.

Upon completion, the reaction was quenched with water (3.0 L), held for 30 minutes and then warmed to 40° C. Aqueous sodium hydroxide (170 g in 1.25 L water) was added over 1.25 hours to bring the pH to 10-11. The mixture was stirred for an hour, then cooled to room temperature and held for 3 hours. The resulting precipitates were filtered and washed with water (100 mL) and heptane (100 mL). The wet solids were heated to reflux (70-75° C.) in acetonitrile:THF and the solution cooled over 3 hours to room temperature. The product was filtered and washed with cold acetonitrile:THF. The product was dried (40-50° C., 10 mm Hg, 24 hours) to give 83% uncorrected yield.

### EXAMPLE 6a

In an analogous synthesis of (E)-N-{4-[3-chloro-4-(3-fluorobenzyloxy)anilino]-3-cyano-7-ethoxy-6-quinolinyl}-4-(dimethylamino)-2-butenamide, a solution of 4-N,N-dimethylaminocrotonic acid hydrochloride (108 g, 0.65 mole) in tetrahydrofuran (1.13 L) and a catalytic amount of dimethylformamide (1.2 mL) was cooled to 0-5° C. Oxalyl chloride (55 mL, 0.62 mole, 0.95 eq) was added dropwise over 50 min. The mixture was then warmed to 25-30° C. and stirred for 2 h then cooled to 0-5° C. N-methyl-2-pyrrolidinone (0.225 L) was added over 25 min followed by a solution of 6-amino-4-[3-chloro-4-(3-fluorobenzyloxy)]anilino-3-cyano-7-ethoxy-quinoline (150 g, 0.32 mol) in N-methyl-2-pyrrolidinone (1.20 L) added dropwise over 2 hours keeping the temperature 0-5° C. The mixture was stirred for at least about 3 hours, warmed to 10-15° C. and stirred for a further 12 hours. The mixture is cooled to 0-10° C., quenched by adding water (1.8 L) over 2 hours, and stirred for 30 minutes. The mixture is warmed to 40° C. Aqueous sodium hydroxide (101 g in 0.75 L water) was added over 1 hour to bring the pH to 10-11. The mixture was stirred for an hour, filtered warm (40° C.) and washed with water (2×0.30 L) until the pH of the last wash was about 7. The wet solids were recrystallized by heating to reflux (70-75° C.) in 60:40 acetonitrile:tetrahydrofuran (2.25 L) and the solution cooled over 3 hours to room temperature. The product was filtered and washed with cold 60:40 acetonitrile:tetrahydrofuran (2×0.30 L). The product was dried (40-50° C., 10 mm Hg, 16 h) to give 0.154 kg (83% yield).

### EXAMPLE 6b

To prepare (E)-N-{4-[4-(benzyloxy)-3-chloroanilino]-3-cyano-7-ethoxy-6-quinolinyl}-4-(dimethylamino)-2-butenamide free base, a solution of 4-N,N-dimethylaminocrotonic acid hydrochloride (18.6 g, 112 mmole) in acetonitrile (295 ml) and a catalytic amount of dimethylformamide (0.25 mL) was cooled to 0-5° C. Oxalyl chloride (9.3 mL, 106 mmole, 0.95 eq) was added dropwise over 5 min. The mixture was then warmed to 25-30° C. and stirred for 1-1.5 h then cooled to 0-10° C. A solution of 6-amino-4-[4-(benzyloxy)-3-chloroanilino]-3-cyano-7-ethoxy-quinoline (25 g, 56 mmole) in N-methyl-2-pyrrolidinone (175 ml) was added dropwise over 30 min keeping the temperature 0-10° C. The mixture was stirred for a minimum of 1 h at 0-10° C. After reaction completion, the mixture was quenched by dropwise addition to a solution of sodium bicarbonate (69.7 g in 870 ml water) over 30 mins. and stirred overnight while warming to room temperature. The mixture was filtered and washed with water (3×25 ml). The crude product was recrystallized in refluxing (80-82° C.) acetonitrile (570 ml). The product was dried (45-50° C., 10 mm Hg, 28 h) to give 12.81 g (41% yield). ¹H NMR: δ (DMSO-d6) 9.44 (s, 1H, NH), 8.97 (s, 1H, Ar), 8.44 (s, 1H, Ar), 7.53-7.35 (m, 7H, Ar), 7.35-7.10 (m, 2H, Ar), 6.78 (dt, 1H, —CH₂CH═CH—), 6.59 (d, 1H, —CH₂CH═CH—), 5.21 (s, 2H, OCH₂Ph), 4.30 (q, 2H, OCH₂CH₃), 3.07 (s, 2H, NCH₂), 2.18 (s, 6H, N(CH₃)₂), 1.47 (t, 3H, OCH₂CH₃).

Results obtained with different reaction procedures at different degrees of scale-up for synthesis of the 2-pyridylmethoxy analog are shown in Table 5.

TABLE 5
Side Chain Coupling

| Scale | | Yield | | |
|---|---|---|---|---|
| (g) | Solvent | (%) | Purity LC (%) | Comments |
| 1 | ACN | 64 | 84 | add acid chloride to aniline, |
| | | | | NaHCO₃ quench |
| 10 | THF | 88 | 97 | reverse addition, NaHCO₃ |
| | | | | quench |
| 10 | THF | 86 | 99 | water, NaOH quench |
| 150 | THF | 68 | TI 2.02* | water, 40° C., NaOH quench, |
| | | | | fast filtration |
| 250 | THF | 85 | 98 | water, 40° C., NaOH quench, |
| | | | | fast filtration |
| 300 | THF | 87 | 99 | water, 40° C., NaOH quench, |
| | | | | fast filtration |
| 250 | THF | 90 | 99 | water, 40° C., NaOH quench, |
| | | | | fast filtration |
| 150 | THF | 83 | 98.8 | water, 40° C., NaOH quench, |
| | | | | fast filtration, 4% SM |
| | | | | remained |

*TI = total impurities

Purificatiuon of the product is conducted by recrystallization in a suitable solvent followed by reslurrying with water followed by additional recrystallization, as necessary. As noted in Table 6, in the synthesis of the 2-pyridylmethoxy analog, several trials in different solvents did not result in the isolation of a single polymorphic form of the product.

TABLE 6

| Scale (g) | Solvent | Volumes | Yield (%) | Comments |
|---|---|---|---|---|
| 4.00 | 2:5:5 | 12 | 97 | Crystallization ˜42° C. |
| | H₂O:THF:ACN | | | |
| 1.00 | 1:5 H₂O:THF | 12 | 84 | DSC 125° C., 183° C. |
| 1.00 | 1:10:5 | 16 | 93 | DSC 122° C., 190° C. |
| | H₂O:THF:EtOAc | | | |
| 1.00 | 11:10 | 12 | 91 | DSC 104° C., 188° C. |
| | DMSO:EtOAc | | | |
| 1.00 | 1:4 | 50 | 92 | DSC 109° C., 180° C., 188° C. |
| | EtOAc:MTBK | | | |
| 1.00 | 1:4 EtOAc:MEK | 50 | 92 | DSC 182° C., 189° C. |
| 1.00 | MIBK | 22 | 91 | DSC 180° C., 187° C. |
| 1.00 | 1:3 EtOAc:THF | 40 | 94 | DSC 119° C., 186° C. |
| 1.00 | 10:7 EtOAc:MeOH | 17 | 78 | DSC 120° C., 189° C. |
| 3.00 | EtOAc | 133 | 87 | |
| 1.00 | MIBK | 40 | 98 | DSC 101° C., 179° C., 186° C. |
| 1.00 | 1:4 EtOAc:MIBK | 50 | 92 | DSC 179° C., 187° C. |
| 2.00 | 1:4 EtOAc:MIBK | 50 | 93 | |
| 1.00 | Acetone | 100 | 80 | |

### EXAMPLE 7

### Formation of Salt

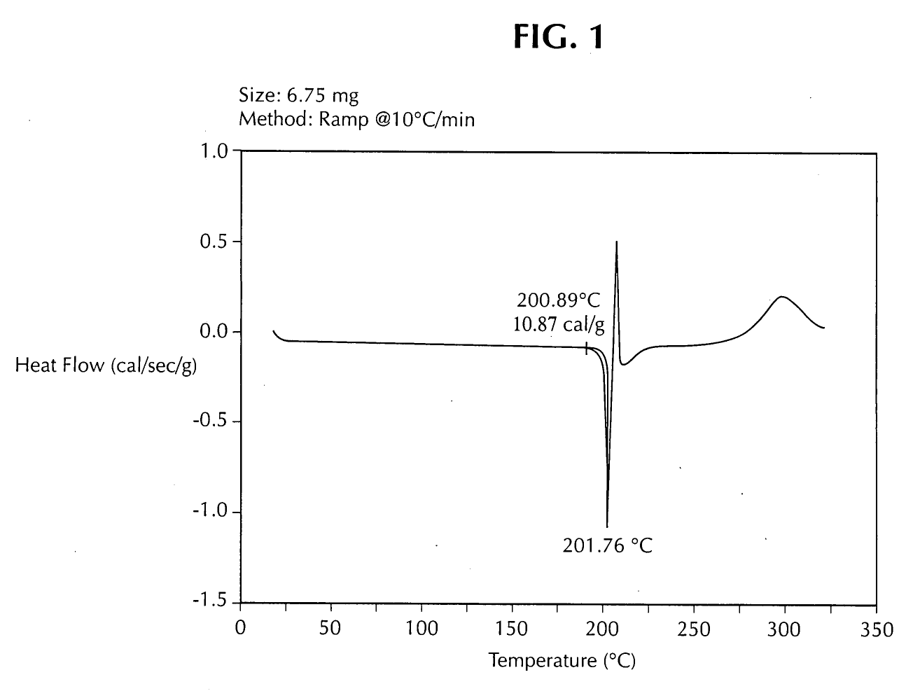

The free base is hygroscopic and undergoes hydrolysis readily. Forming a salt of the compound, such as a flumarate or mesylate salt, stabilizes the molecule and renders the compound more soluble. The most preferred salt is a maleate salt, which has been found to be highly crystalline and to exist substantially as a single polymorph as shown by DSC thermogram in FIG. 1.

Recrystallizing the product in the presence of an acid has been found to yield a stable salt form of the product. Experimental results achieved utilizing different solvents for the recrystallization are set forth in Table 7. As seen in Table 7, an improvement is observed when n-propanol/water is used as the solvent system. A maleate salt is the most preferred, as it exists in a single polymorphic form.

TABLE 7
Recrystallization

| Scale (g) | Solvent (vols) | H₂O (vols) | Yield (%) |
|---|---|---|---|
| 2.45 | EtOAc (50) | 0 | 62 |
| 1.97 | n-BuOH (50) | 0 | 66 |
| 1.00 | EtOH 1L (10) | 1 | 27 |
| 0.50 | EtOAc (140) | 0 | 88 |
| 0.50 | EtOH 1L (20) | 0 | 100 |
| 0.25 | EtOAc:MeOH | 0 | — |
| | (100:100) | | |
| 3.24 | EtOH 1L (32) | 0 | 84 |
| 1.00 | EtOH 1L (15) | 1 | 83 |
| 1.00 | MeOH:EtOAc | 0 | 86 |
| | (2:3) | | |
| 1.00 | EtOH 1L (15) | 1 | 69 |
| 2.00 | MeOH:EtOAc | 0 | 91 |
| | (13.9:9.3) | | |
| 4.00 | MeOH:EtOAc | 0 | 83 |
| | (13.9:9.3) | | |
| 1.00 | IPA (15) | 4 | 86 |
| 1.00 | IPA (13.5) | 1.5 | 90 |
| 1.00 | IPA (13.5) | 1.5 | 84 |
| 1.00 | IPA (18) | 2 | 86 |
| 1.00 | n-PrOH (18) | 2 | 63 |
| 1.00 | n-PrOH (9) | 1 | 83 |
| 1.00 | n-PrOH (13.5) | 1.5 | 78 |
| 9.00 | n-PrOH (10.8) | 1.2 | 72 |
| 4.75 | n-PrOH (12.6) | 1.4 | 83 |
| 5.00 | n-PrOH (10.8) | 1.2 | 91 |
| 5.00 | n-PrOH (10.8) | 1.2 | 85 |
| 5.00 | n-PrOH (10.8) | 1.2 | 78 |
| 40 | n-PrOH (10.8) | 1.2 | 80 |
| 375 | n-PrOH (10.8) | 1.2 | 90 |
| 100 | n-PrOH (10.8) | 1.2 | 88 |

Preparation of (E)-N-{4-[3-chloro-4-(2-pyridinylmethoxy)anilino]-3-cyano-7-ethoxy-6-quinolinyl}-4-(dimethylamino)-2-butenamide maleate, WAY-179272-B

(E)-N-{4-[3-chloro-4-(2-pyridinylmethoxy)anilino]-3-cyano-7-ethoxy-6-quinolinyl}-4-dimethylamino)-2-butenamide crude free base (0.1 kg, 0.159 mole) and maleic acid (0.019 kg, 0.164 mole) were dissolved at 40-50° C. in a 10% water/n-propanol mixture (1.20 L). The hot solution was clarified and cooled over 2 h to room temperature and held for 12-15 hr. The product was filtered and washed with 10% water/n-propanol (2×0.15 L). The product was dried (50° C., 10 mm Hg, 24 h) to give 94.4 g (88% yield). DSC: 204° C. (single crystal form). ¹H NMR: δ (DMSO-d6) 9.73 (s, 1H, NH), 9.62 (s, 1H, NH), 8.93 (s, 1H, Ar), 8.60 (dd, 1H, Ar), 8.50 (s, 1H, Ar), 7.88 (dd, 1H, Ar), 7.58 (d, 1H, Ar), 7.40 (m, 3H, Ar), 7.24 (m, 2H, Ar), 6.75 (d, 2H, —CH═CH—), 6.03 (s, 2H, HOOC—CH═CH—COOH), 5.29 (s, 2H, OCH₂Pyr), 4.33 (q, 2H, OCH₂CH₃), 3.89 (s, 2H, NCH₂), 2.76 (s, 6H, N(CH₃)₂), 1.47 (t, 3H, OCH₂CH₃). ¹³C NMR: δ (DMSO-d6) 168.0, 163.2, 156.9, 154.2, 153.2, 151.9, 151.3, 149.8, 148.5, 137.8, 136.5, 134.7, 133.4, 132.2, 128.0, 126.6, 124.9, 123.8, 122.3, 122.2, 117.9, 116.4, 115.1, 113.9, 109.5, 88.1, 72.0, 65.3, 57.8, 43.1, 14.9.

### EXAMPLE 7a

To prepare (E)-N-{4-[3-chloro-4-(3-fluorobenzyloxy)anilino]-3-cyano-7-ethoxy-6-quinolinyl}-4-(dimethylamino)-2-butenamide dimaleate, (E)-N-{4-[3-chloro-4-(3-fluorobenzyloxy)anilino]-3-cyano-7-ethoxy-6-quinolinyl}-4-dimethylamino)-2-butenamide crude free base (0.516 kg, 0.90 mole) and maleic acid (0.214 kg, 1.84 mole) were dissolved at 40-50° C. in a 6.5% water/n-propanol mixture (12.60 L). The hot solution was clarified, rinsed with 5% water/n-propanol (0.52 L) and n-propanol (2.0 L). The mixture was held at 45° C. for 3 hr, cooled over 2 h to room temperature and held overnight. The mixture was further cooled to 5-10° C. The product was filtered and washed with cold 5% water/n-propanol (0.52 L). The product was dried (45° C., 10 mm Hg, 16-24 h) to give 0.586 kg (81% yield). DSC: 184° C. (single crystal form). ¹H NMR: δ (DMSO-d6) 9.77 (s, 1H, NH), 8.95 (s, 1H, Ar), 8.53 (s, 1H, Ar), 7.49-7.16 (m, 8H, Ar), 6.78 (m, 2H, —CH═CH—), 6.15 (s, 4H, 2× HOOC—CH═CH—COOH), 5.26 (s, 2H, OCH₂Pyr), 4.33 (q, 2H, OCH₂CH₃), 3.97 (dd, 2H, NCH₂), 2.82 (s, 6H, N(CH₃)₂), 1.47 (t, 3H, OCH₂CH3). ¹³C NMR: δ (DMSO-d6) 167.0, 163.8, 162.3, 160.6, 153.6, 152.2, 151.3, 150.8, 139.5, 139.4, 133.7, 133.2, 132.2, 131.8, 130.5, 130.4, 127.4, 126.1, 124.3, 123.3, 121.7, 116.9, 115.7, 114.8, 114.5, 114.4, 114.1, 113.8, 113.1, 108.1, 87.2, 69.5, 64.6, 56.9, 42.1, 14.2.

### EXAMPLE 7b

To prepare (E)-N-{4-[4-(benzyloxy)-3-chloroanilino]-3-cyano-7-ethoxy-6-quinolinyl}-4-(dimethylamino)-2-butenamide maleate, (E)-N-{4-[4-(benzyloxy)-3-chloroanilino]-3-cyano-7-ethoxy-6-quinolinyl}-4-dimethylamino)-2-butenamide crude free base (2.0 g, 3.6 mmole) and maleic acid (0.43 g, 3.7 mmole) were mixed at 40-50° C. in a 10% water/n-propanol mixture (24 ml) for 2 hr. The mixture was cooled to ambient temperature, filtered and washed with 10% water/n-propanol (2×3 ml). The product was dried (40° C., 10 mm Hg, 24 h) to give 0.32 g (13% yield). ¹H NMR: δ (DMSO-d6) 9.75 (s, 1H, NH), 8.95 (s, 1H, Ar), 8.49 (s, 1H, Ar), 7.49-7.37 (m, 7H, Ar), 7.23 (dd, 2H, Ar), 6.78 (s, 2H, —CH₂CH═CH—), 6.06 (s, 2H, HOOC—CH═CH—COOH), 5.22 (s, 2H, OCH₂Ph), 4.31 (q, 2H, OCH₂CH₃), 3.93 (s, 2H, NCH₂), 2.79 (s, 6H, N(CH₃)₂), 1.46 (t, 3H, OCH₂CH₃). ¹³C NMR: δ (DMSO-d6) 167.9, 163.1, 154.2, 153.3, 152.1, 151.3, 148.5, 137.3, 136.3, 134.5, 133.2, 132.3, 129.3, 129.2, 128.7, 128.3, 128.2, 128.0, 126.7, 124.9, 122.4, 117.9, 116.4, 115.2, 113.9, 109.5, 88.0, 71.1, 65.3, 57.7, 43.0, 15.0.

(E)-N-{4-[4-(benzyloxy)-3-chloroanilino]-3-cyano-7-ethoxy-6-quinolinyl}-4-dimethylamino)-2-butenamide crude free base (2.0 g, 3.6 mmole) and maleic acid (0.43 g, 3.7 mmole) were mixed at 40-50° C. in a 10% water/n-propanol mixture (24 ml) for 2 hr. The mixture was cooled to ambient temperature, filtered and washed with 10% water/n-propanol (2×3 ml). The product was dried (40° C., 10 mm Hg, 24 h) to give 0.32 g (13% yield). ¹H NMR: δ (DMSO-d6) 9.75 (s, 1H, NH), 8.95 (s, 1H, Ar), 8.49 (s, 1H, Ar), 7.49-7.37 (m, 7H), Ar), 7.23 (dd, 2H, Ar), 6.78 (s, 2H, —CH₂CH═CH—), 6.06 (s, 2H, HOOC—CH═CH—COOH), 5.22 (s, 2H, OCH₂Ph), 4.31 (q, 2H, OCH₂CH₃), 3.93 (s, 2H, NCH₂), 2.79 (s, 6H, N(CH₃)₂), 1.46 (t, 3H, OCH₂CH₃). ¹³C NMR: δ (DMSO-d6) 167.9, 163.1, 154.2, 153.3, 152.1, 151.3, 148.5, 137.3, 136.3, 134.5, 133.2, 132.3, 129.3, 129.2, 128.7, 128.3, 128.2, 128.0, 126.7, 124.9, 122.4, 117.9, 116.4, 115.2, 113.9, 109.5, 88.0, 71.1, 65.3, 57.7, 43.0, 15.0.

The scope of the invention is not limited by the embodiments disclosed herein. Variations and modifications of the methods disclosed will be apparent to those of ordinary skill in the art and are within the scope of the invention defined by the following claims.

What is claimed is:

1. A method for preparing substituted 3-cyanoquinolines comprising the step of reacting
(i) a compound of formula H-Z-CH₂)ₙ—X, and
(ii) a 3-cyanoquinoline intermediate having formula (Ia) 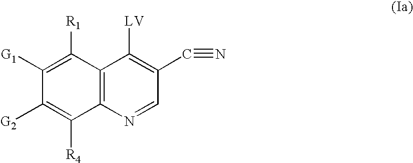
in the presence of a catalytic effective amount of an acid catalyst to produce a compound of formula (IIa) 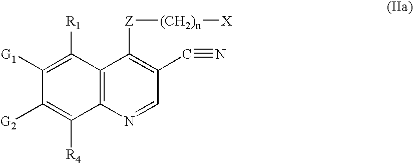
wherein X is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkyl amino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or X is cycloalkyl of 3 to 7 carbon atoms, which may be optionally substituted with one or more alkyl of 1 to 6 carbon atom groups; or
X is a pyridinyl, pyrimidinyl, or phenyl ring, wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino; or
X is a radical having the formula: 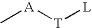
wherein A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
T is bonded to a carbon of A and is:
—NH(CH₂)ₘ—, —O(CH₂)ₘ—, —S(CH₂)ₘ—, —NR(CH₂)ₘ—, —(CH₂)ₘ—, —(CH₂)ₘNH—, —(CH₂)ₘO—, —(CH₂)ₘS—, or —(CH₂)ₘNR—;
L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
LV is a leaving group,
Z is —NH—, —O—, —S—, or —NR—,
R is alkyl of 1-6 carbon atoms,
G₁, G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 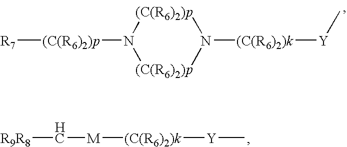
R₇—(C(R₆)₂)_{g}—Y—, R₇—(C(R₆)₂)ₚ-M-(C(R₆)₂)ₖ—Y—, or Het-(C(R₆)₂)_{q}—W—(C(R₆)₂—Y—; or optionally
G₁ and/or G₂ are independently selected from a protected amino group and R₂NH—;
Y is a divalent radical selected from the group consisting of 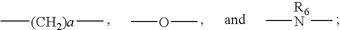
R₇ is —NR₆R₆, —OR₆, -J, —N(R₆)₃⁺, or —NR₆(OR₆);
M is >NR₆, —O—, >N—(C(R₆)₂)ₚNR₆R₆, or >N—(C(R₆)₂)ₚ—OR₆;
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 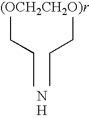
wherein Het is optionally mono- or di-substituted on carbon or nitrogen with R₆, optionally mono- or di-substituted on carbon with hydroxy, —N(R₆)₂, or —OR₆, optionally mono or di-substituted on carbon with the mono-valent radicals —(C(R₆)₂)ₛOR₆ or —(C(R)₂)ₛN(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂ is selected from the group consisting of 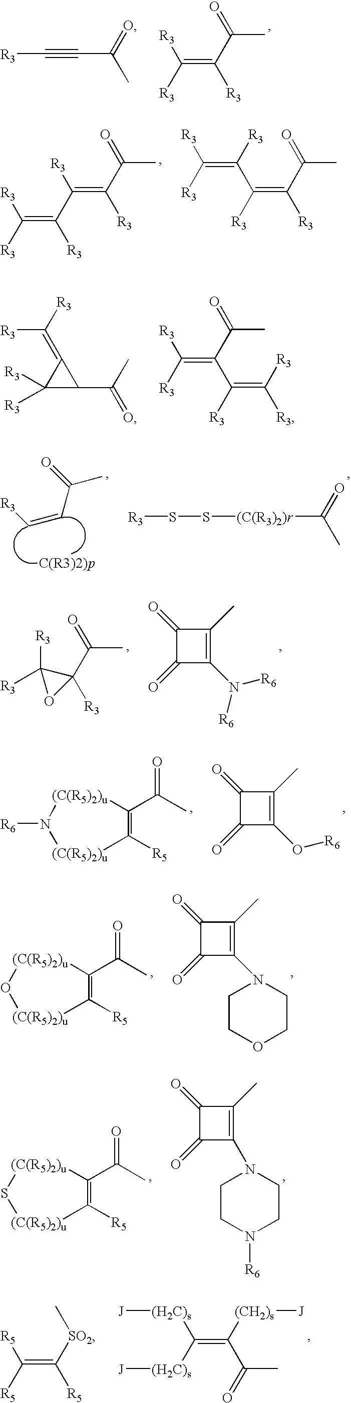 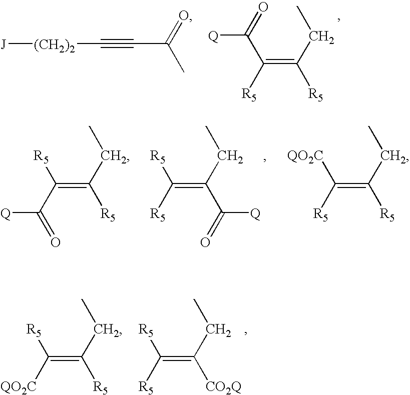
R₃ is independently hydrogen, alkyl of 1-6 carbon atoms, aminoalkyl of 1-6 carbon atoms, cycloaminoalkyl of 4-12 carbon atoms, carboxy, carboalkoxy of 1-6 carbon atoms, phenyl, carboalkyl of 2-7 carbon atoms, 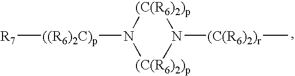
R₇—(C(R₆)₂)ₛ—, R₇—(C(R₆)₂)ₚ-M-(C(R₇)₂)ᵣ—, R₈R₉—CH-M-(C(R₆)₂)ᵣ—, or Het-(C(R₆)₂)_{q}—W—(C(R₆)₂)ᵣ—;
R₅ is independently hydrogen, alkyl of 1-6 carbon atoms, carboxy, carboalkoxy of 1-6 carbon atoms, phenyl, carboalkyl of 2-7 carbon atoms, 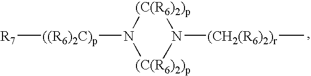
R₇—(C(R₆)₂)ₛ—, R₇—(C(R₆)₂)ₚ-M-(C(R₆)₂)ᵣ—, R₈R₉—CH-M-(C(R₆)₂)ᵣ—, or Het-(C(R₆)₂)_{q}—W—(C(R₆)₂)ᵣ—;
R₈, and R₉ are each, independently, —(C(R₆)₂)ᵣNR₆R₆, or —(C(R₆)₂)ᵣOR₆;
J is independently hydrogen, chlorine, fluorine, or bromine;
Q is alkyl of 1-6 carbon atoms or hydrogen;
a=0 or 1;
g=1-6;
k=0-4;
m is 0-3;
n is 0-1,
p=2-4;
q=0-4;
r=1-4;
s=1-6;
u=0-4 and v=0-4, wherein the sum of u+v is 2-4.

2. The method according to claim 1, wherein G₁ is a protected amino group selected from the group consisting of acetamides, benzamides, cyclic imides, pyrroles, tert-butoxycarbonyl protected amine and benzyloxycarbonyl protected amine.

3. A method for preparing 4-amino-3-cyanoquinolines comprising the step of reacting
(i) a compound of formula H₂N—(CH₂)ₙ—X, and
(ii) a 3-cyanoquinoline starting material having formula (I) 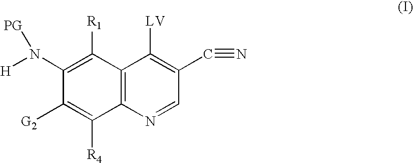
in the presence of a catalytic effective amount of an acid catalyst to produce a 4-amino-3-cyanoquinoline having formula (II) 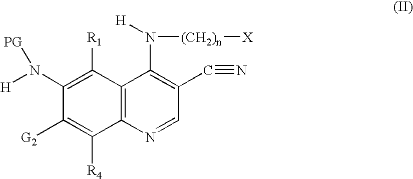
wherein X is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkyl amino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or X is cycloalkyl of 3 to 7 carbon atoms, which may be optionally substituted with one or more alkyl of 1 to 6 carbon atom groups; or
X is a pyridinyl, pyrimidinyl, or phenyl ring, wherein the pyridinyl, pyrimidinyl, or phenyl ring optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino; or
X is a radical having the formula: 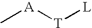
wherein A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
T is bonded to a carbon of A and is:
—NH(CH₂)ₘ—, —O(CH₂)ₘ—, —S(CH₂)ₘ—, —NR CH₂)ₘ—, —(CH₂)ₘ—, —(CH₂)ₘNH—, —(CH₂)ₘO—, —(CH₂)ₘS—, or —(CH₂)ₘNR—;
L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; provided that L can be an unsubstituted phenyl ring only when m>0 and T is not —CH₂—NH—; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
LV is a leaving group,
PG is a protecting group,
G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 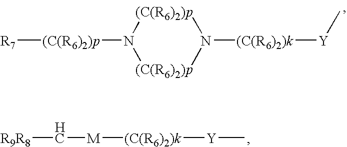
R₇—(C(R₆)₂)_{g}—Y—, R₇—(C(R₆)₂)ₚ-M-(C(R₆)₂)ₖ—Y—, or
Het-(C(R₆)₂)_{q}W—(C(R₆)₂—Y—; or R₁ and R₄ are as defined above and G₂ is R₂—NH—;
Y is a divalent radical selected from the group consisting of 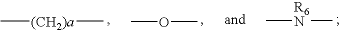
R₇ is —NR₆R₆, —OR₆, -J, —N(R₆)₃⁺, or —NR₆(OR₆);
M is >NR₆, —O—, >N—(C(R₆)₂)ₚNR₆R₆, or >N—(C(R₆)₂)ₚ—OR₆;
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 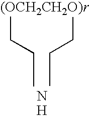
wherein Het is optionally mono- or di-substituted on carbon or nitrogen with R₆, optionally mono- or di-substituted on carbon with hydroxy, —N(R₆)₂, or —OR₆, optionally mono or di-substituted on carbon with the mono-valent radicals —(C(R₆)₂)ₛOR₆ or —(C(R₆)₂)ₛN(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂ is selected from the group consisting of 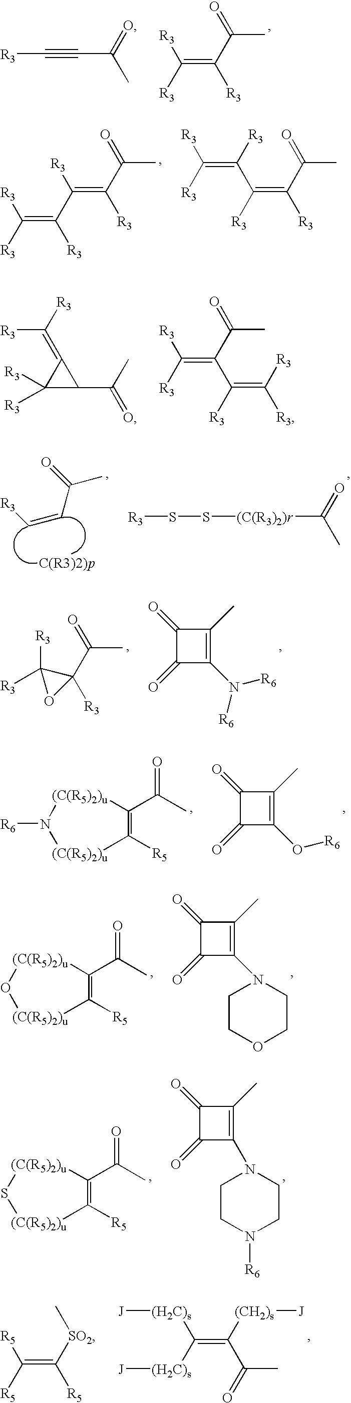 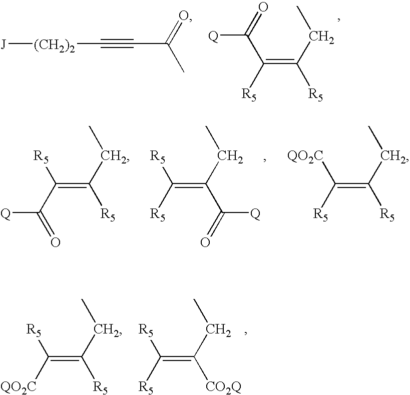
R₃ is independently hydrogen, alkyl of 1-6 carbon atoms, aminoalkyl of 1-6 carbon atoms, cycloaminoalkyl of 4-12 carbon atoms, carboxy, carboalkoxy of 1-6 carbon atoms, phenyl, carboalkyl of 2-7 carbon atoms, 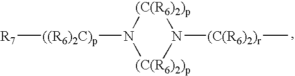
R₇—(C(R₆)₂)ₛ—, R₇—(C(R₆)₂)ₚ-M-(C(R₇)₂)ᵣ—, R₈R₉—CH-M-(C(R₆)₂)ᵣ—, or Het-(C(R₆)₂)_{q}—W—(C(R₆)₂)ᵣ—;
R₅ is independently hydrogen, alkyl of 1-6 carbon atoms, carboxy, carboalkoxy of 1-6 carbon atoms, phenyl, carboalkyl of 2-7 carbon atoms, 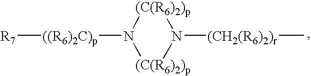
R₇—(C(R₆)₂)ₛ—, R₇—(C(R₆)₂)ₚ⁻M-(C(R₆)2)ᵣ—, R₈R₉—CH-M-(C(R₆)₂)ᵣ—, or Het-(C(R₆)₂)_{q}—W—(C(R₆)₂)ᵣ—;
R₈, and R₉ are each, independently, —(C(R₆)₂)ᵣNR₆R₆, or —(C(R₆)₂)ᵣOR₆;
J is independently hydrogen, chlorine, fluorine, or bromine;
Q is alkyl of 1-6 carbon atoms or hydrogen;
a=0 or 1;
g=1-6;
k=0-4;
n is 0-1;
m is 0-3;
p=2-4;
q=0-4;
r=1-4;
s=1-6;
u=0-4 and v=0-4, wherein the sum of u+v is 2-4.

4. The method according to claim 1, wherein
X is a phenyl ring, optionally mono-, di- or tri-substituted with halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, or benzoylamino; or
X is a radical defined by -A-T-L, wherein
A is phenyl ring which is unsubstituted or mono- or di-substituted with halogen,
T is bonded to a carbon of A and is —O(CH₂)ₘ—, and
L is an unsubsitituted phenyl ring or a phenyl ring mono-, di-, or tri-substituted with halogen; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with halogen,
R₁ and R₄ are hydrogen,
G₂ is alkoxy,
n=0, and
m=1.

5. The method according to claim 3, further comprising the step of deprotecting the 4-amino-3-cyanoquinoline of formula (II) to form a 4-amino-3-cyanoquinoline of formula (III) 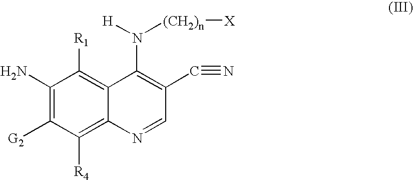
wherein n, X, R₁, R₄ and G₂ are as defined in claim 1.

6. The method according to claim 5, wherein said step of deprotecting is conducted without isolating the 4-amino-3-cyanoquinoline of formula (II).

7. The method according to claim 5, further comprising the step of reacting the 4-amino-3-cyanoquinoline of formula (III) with a carboxylic acid chloride of formula 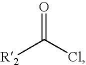
or a mixed anhydride of a corresponding carboxylic acid, to form a 4-amino-3-cyanoquinoline of formula (A′) 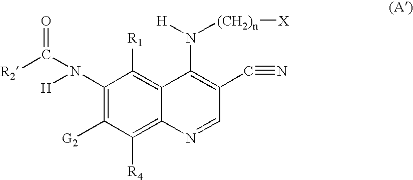
wherein R′₂ is alkyl of 1-6 carbon atoms, optionally mono or di-substituted with amino groups or cycloamino groups, or R′₂ is alkenyl of 2-6 carbon atoms optionally mono or di-substituted with amino groups or cycloamino groups.

8. A method according to claim 3, wherein said compound of formula H₂N—(CH₂)ₙ—X is formed such that n is 0 and X is Ar—O—CH₂-L′, by the steps of
(a′) reacting Ar—NO₂ with L′-CH₂)—OH to form a nitroaryl intermediate NO₂—Ar OCH₂-L′, and
(a″) catalytically hydrogenating the nitroaryl intermediate of step (a′) to form a first aniline intermediate NO₂—Ar—OH₂-L′,
wherein Ar is a pyridinyl, pyrimidinyl, or phenyl ring, wherein the pyridinyl, pyrimidinyl, or phenyl ring optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino; and
L′ is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
L′ is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino.

9. The method of claim 8, wherein said first aniline intermediate formed in step (a″) is an aryloxy aniline selected from the group consisting of 3-chloro-4-(pyridylmethoxy)aniline, 3-chloro-4-(benzyloxy)aniline, 3-chloro-4-(fluorobenzyloxy)aniline, and 3-chloro-4-(thiophenyl)aniline.

10. A method of synthesizing substituted 3-cyanoquinolines comprising the steps of:
reacting an activated carboxylate of formula (VI) 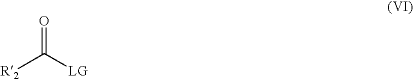
with an intermediate of formula (III′) 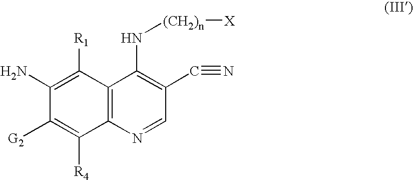
to form a compound of formula (VII) 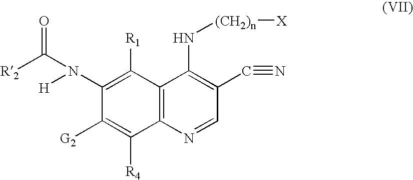
wherein, LG is a leaving group such that formula (VI) is an activated carboxylate selected from the group consisting of halide, anhydride, acyl azide, 1,3,5-triazine, aromatic boronic acid, Lawesson's reagent, peptide-type coupling reagent, DCC, TiCl₄, activated phosphate, Sn[N(TMS)₂]₂, N-halosuccinimide/Ph₃P, Cl₃CCN/Ph₃P, (R₂N)₂Mg, SO₂ClF, chlorosulfonyl isocyanide, TsCl/base, metal alkoxides, PyBOP, BOP, and EDCI/HOBt
R′₂ is alkyl of 1-6 carbon atoms, optionally mono or di-substituted with amino groups or cycloamino groups, or R′₂ is alkenyl of 2-6 carbon atoms optionally mono or di-substituted with amino groups or cycloamino groups;
wherein X is a pyridinyl, pyrimidinyl, or phenyl ring, wherein the pyridinyl, pyrimidinyl, or phenyl ring optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino; or
X is a radical having the formula: 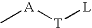
wherein A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
T is bonded to a carbon of A and is:
—NH(CH₂)ₘ—, —(CH₂)ₘ—, —S(CH₂)ₘ—, —NR(CH₂)ₘ—, —(CH₂)ₘ—, —(CH₂)ₘNH—, —(CH₂)ₘO—, —(CH₂)ₘS—, or —(CH₂)ₘNR—;
L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
and wherein G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 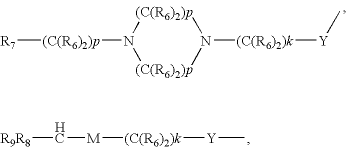
R₇—(C(R₆)₂)_{g}—Y—, R₇—(C(R₆)₂)ₚ-M-(C(R₆)₂)ₖ—Y—, or Het-(C(R₆)₂)_{q}W—(C(R₆)₂—Y—; or optionally
G₂ is selected from a protected amino group and R₂—NH—;
Y is a divalent radical selected from the group consisting of 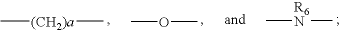
R₇ is —NR₆R₆, —OR₆, -J, —N(R₆)₃⁺, or —NR₆(OR₆);
M is >NR₆, —O—, >N—(C(R₆)₂)ₚNR₆R₆, or >N—(C(R₆)₂)ₚ—OR₆;
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 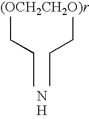
wherein Het is optionally mono- or di-substituted on carbon or nitrogen with R₆, optionally mono- or di-substituted on carbon with hydroxy, —N(R₆)₂, or —OR₆, optionally mono or di-substituted on carbon with the mono-valent radicals —(C(R₆)₂)ₛOR₆ or —(C(R₆)₂)ₛN(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂ is selected from the group consisting of 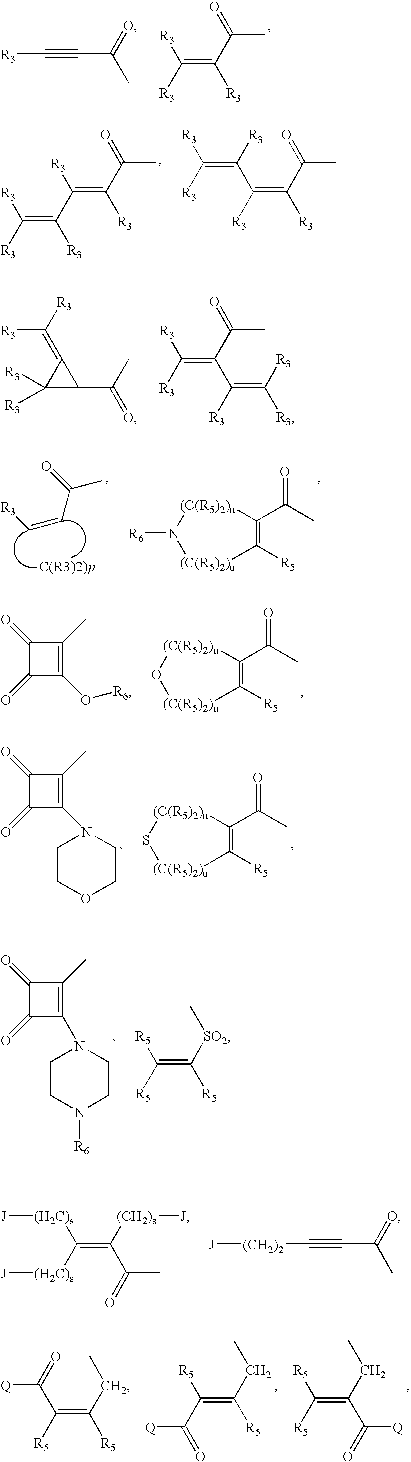 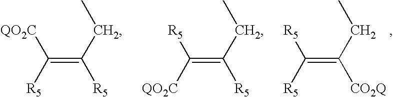
R₃ is independently hydrogen, alkyl of 1-6 carbon atoms, aminoalkyl of 1-6 carbon atoms, cycloaminoalkyl of 4-12 carbon atoms, carboxy, carboalkoxy of 1-6 carbon atoms, phenyl, carboalkyl of 2-7 carbon atoms, 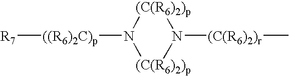
R₇—(C(R₆)₂)ₛ—, R₇—(C(R₆)₂)ₚ-M-(C(R₇)₂)ᵣ—, R₈R₉—CH-M-(C(R₆)₂)ᵣ—, or Het-(C(R₆)₂)_{q}—W—(C(R₆)₂)ᵣ—;
R₅ is independently hydrogen, alkyl of 1-6 carbon atoms, carboxy, carboalkoxy of 1-6 carbon atoms, phenyl, carboalkyl of 2-7 carbon atoms, 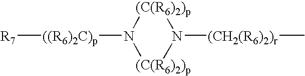
R₇—(C(R₆)₂)ₛ—, R₇—(C(R₆)₂)ₚ-M-(C(R₆)₂)ᵣ—, R₈R₉—CH-M-(C(R₆)₂)ᵣ—, or Het-(C(R₆)₂)_{q}—W—(C(R₆)₂)ᵣ—;
R₈, and R₉ are each, independently, —(C(R₆)₂)ᵣNR₆R₆, or —(C(R₆)₂)ᵣOR₆;
J is independently hydrogen, chlorine, fluorine, or bromine;
Q is alkyl of 1-6 carbon atoms or hydrogen;
a=0 or 1;
g=1-6;
k=0-4;
m is 0-3;
n is 0-1;
p=2-4;
q=0-4;
r=1-4;
s=1-6;
u=0-4 and v=0-4, wherein the sum of u+v is 2-4.

11. The method of claim 10, further comprising the step of recrystallizing said compound (VII) from a mixture of said compound (VII) in a solvent to form a salt.

12. The method of claim 10, wherein R′2 is is a 4-(dimethylamino)-2-butenyl radical, a 4-(piperidino)-2-butenyl radical, a 4-(pyrrolidino)-2-butenyl radical, or a 3,4-(dipyrrolidino)-2-butenyl radical.

13. The method of claim 10, wherein n=0 and X is a 3-chloro-4-(pyridylmethoxyl)phenyl radical, a 3-chloro-4-(benyloxy)phenyl radical, a 3-chloro-4-(fluorobenzyloxy)phenyl radical, or a 3-chloro-4-(thiophenyl)phenyl radical.

14. The method of claim 13, wherein said compound (VII) is recrystallized in the presence of maleic acid to form a maleate salt of said compound.
